SEQUENCE LISTING (12 sequences)

SEQ ID NO: 1 - DNA, 1625 bp, Homo sapiens
  CDS (45)..(1622)
tgtgctgtagtgagaggctgttgggtcgcccccagccgatagtgatggaggcactggacgaggctgaagggctccaggactcacagagagagatgccgccaccccctcctccctcgccgccctcagatccagctcagaagccaccacctcgaggcgctgggagccactccctcactgtcaggagcagcctgtgcctgttcgctgcctcacagttcctgcttgcctgtggggtgctctggttcagcggttatggccacatctggtcacagaacgccacaaacctcgtctcctctttgctgacgctcctgaaacagctggaacccacggcctggcttgactctgggacgtggggagtccccagtctgctgctggtctttctgtccgtgggcctggtcctcgttaccaccctggtgtggcacctcctgaggacacccccagagccacccaccccactgccccctgaggacaggcgccagtcagtgagccgccagccctccttcacctactcagagtggatggaggagaagatcgaggatgacttcctggacctcgacccggtgcccgagactcctgtgtttgattgtgtgatggacatcaagcctgaggctgaccccacctcactcaccgtcaagtccatgggtctgcaggagaggaggggttccaatgtctccctgaccctggacatgtgcactccgggctgcaacgaggagggctttggctatctcatgtccccacgtgaggagtccgcccgcgagtacctgctcagcgcctcccgtgtcctccaagcagaagagcttcatgaaaaggccctggaccctttcctgctgcaggcggaattctttgaaatccccatgaactttgtggatccgaaagagtacgacatccctgggctggtgcggaagaaccggtacaaaaccatacttcccaaccctcacagcagagtgtgtctgacctcaccagaccctgacgaccctctgagttcctacatcaatgccaactacatccggggctatggtggggaggagaaggtgtacatcgccactcagggacccatcgtcagcacggtcgccgacttctggcgcatggtgtggcaggagcacacgcccatcattgtcatgatcaccaacatcgaggagatgaacgagaaatgcaccgagtattggccggaggagcaggtggcgtacgacggtgttgagatcactgtgcagaaagtcattcacacggaggattaccggctgcgactcatctccctcaagagtgggactgaggagcgaggcctgaagcattactggttcacatcctggcccgaccagaagaccccagaccgggcccccccactcctgcacctggtgcgggaggtggaggaggcagcccagcaggaggggccccactgtgcccccatcatcgtccactgcagtgcagggattgggaggaccggctgcttcattgccaccagcatctgctgccagcagctgcggcaggagggtgtggtggacatcctgaagaccacgtgccagctccgtcaggacaggggcggcatgatccagacatgcgagcagtaccagtttgtgcaccacgtcatgagcctctacgaaaagcagctgtcccaccagtccccagaatga

SEQ ID NO: 2 - protein, 526 aa, Homo sapiens
MEALDEAEGLQDSQREMPPPPPPSPPSDPAQKPPPRGAGSHSLTVRSSLCLFAASQFLLACGVLWFSGYGHIWSQNATNLVSSLLTLLKQLEPTAWLDSGTWGVPSLLLVFLSVGLVLVTTLVWHLLRTPPEPPTPLPPEDRRQSVSRQPSFTYSEWMEEKIEDDFLDLDPVPETPVFDCVMDIKPEADPTSLTVKSMGLQERRGSNVSLTLDMCTPGCNEEGFGYLMSPREESAREYLLSASRVLQAEELHEKALDPFLLQAEFFEIPMNFVDPKEYDIPGLVRKNRYKTILPNPHSRVCLTSPDPDDPLSSYINANYIRGYGGEEKVYIATQGPIVSTVADFWRMVWQEHTPIIVMITNIEEMNEKCTEYWPEEQVAYDGVEITVQKVIHTEDYRLRLISLKSGTEERGLKHYWFTSWPDQKTPDRAPPLLHLVREVEEAAQQEGPHCAPIIVHCSAGIGRTGCFIATSICCQQLRQEGVVDILKTTCQLRQDRGGMIQTCEQYQFVHHVMSLYEKQLSHQSPE

SEQ ID NO: 3 - DNA, 1711 bp, Homo sapiens
  CDS (1)..(1614): partial CDS; 5′ end undefined, <1..1614
gagaggctgttgggtcgcccccagccgatagtgatggaggcactggacgaggctgaagggctccaggactcacagagagagatgccgccaccccctcctccctcgccgccctcagatccagctcagaagccaccacctcgaggcgctgggagccactccctcactgtcaggagcagcctgtgcctgttcgctgcctcacagttcctgcttgcctgtggggtgctctggttcagcggttatggccacatgtggtcacagaacgccacaaacctcgtctcctctttgctgacgctcctgaaacagctggaacccacgtcctggcttgactctgggacgtggggagtccccggtctgctgctggtctttctgtccgtgggcctggtcctcgttaccaccctggtgtggcacctcctgaggacacccccagagccacccaccccactgccccctgaggacaggcgccagtcagtgagccgccagccctccttcacctactcagagtggatggaggagaagatcgaggatgacttcctggacctcgacccggtgcccgagactcctgtgtttgattgtgtgatggacatcaagcctgaggctgaccccacctcactcaccgtcaagtccatgggtctgcaggagaggaggggttccaatgtctccctgaccctggacatgtgcactccgggctgcaacgaggagggctttggctatctcatgtccccacgtgaggagtccgcccgcgagtacctgctcagcgcctcccgtgtcctccaagcagaagagcttcatgaaaaggccctggaccctttcctgctgcaggcggaattctttgaaatccccatgaactttgtggtaccgaaagagtacgacatccctgggaggtgtcggaagaaccggtacaaaaccatacttcccaaccctcacagcagagtgtgtctgacctcaccagaccctgacgaccctctgagttcctacatcaatgccaactacatccggggctatggtggggaggagaaggtgtacatcgccactcagggacccatcgtcagcacggtcgccgacttctggcgcatggtgtggcaggagcacacgcccatcattgtcatgatcaccaacatcgaggagatgaacgagaaatgcaccgagtattggccggaggagcaggtggcgtacgacggtgttgagatcactgtgcagaaagtcattcacacggaggattaccggctgcgactcatctccctcaagagtgggactgaggagcgaggcctgaagcattactggttcacatcctggcccgaccagaagaccccagaccgggcccccccactcctgcacctggtgcgggaggtggaggaggcagcccagcaggagggtccccactgtgcccccatcatcgtccactgcagtgcagggattgggaggaccggctgcttcattgccaccagcatctgctgccagcagctgcggcaggagggtgtagtggacatcctgaagaccacgtgccagctccgtcaggacaggggcggcatgatccagcactgcgagcagtaccagtttgtgcaccacgtcatgagcctctacgaaaagcagctgtcccaccagtccccagaatgactgcgcttctcctacaaggttctctgggcactgccagcctgagtctcggccctcacccagggccctgcctcgggtcggaattcgatatcaagcttat

SEQ ID NO: 4 - protein, 537 aa, Homo sapiens
ERLLGRPQPIVMEALDEAEGLQDSQREMPPPPPPSPPSDPAQKPPPRGAGSHSLTVRSSLCLFAASQFLLACGVLWFSGYGHMWSQNATNLVSSLLTLLKQLEPTSWLDSGTWGVPGLLLVFLSVGLVLVTTLVWHLLRTPPEPPTPLPPEDRRQSVSRQPSFTYSEWMEEKIEDDFLDLDPVPETPVFDCVMDIKPEADPTSLTVKSMGLQERRGSNVSLTLDMCTPGCNEEGFGYLMSPREESAREYLLSASRVLQAEELHEKALDPFLLQAEFFEIPMNFVVPKEYDIPGRCRKNRYKTILPNPHSRVCLTSPDPDDPLSSYINANYIRGYGGEEKVYIATQGPIVSTVADFWRMVWQEHTPIIVMITNIEEMNEKCTEYWPEEQVAYDGVEITVQKVIHTEDYRLRLISLKSGTEERGLKHYWFTSWPDQKTPDRAPPLLHLVREVEEAAQQEGPHCAPIIVHCSAGIGRTGCFIATSICCQQLRQEGVVDILKTTCQLRQDRGGMIQHCEQYQFVHHVMSLYEKQLSHQSPE

SEQ ID NO: 5 - DNA, 2965 bp, Homo sapiens
  CDS (296)..(1993)
gcgcacggagcgcgggacggagcgccaggcggacggaccgaaggacggaggcaccgaaggacggacgcccccgcacacgcagacgcacagagctcggcgcggcccccgtcgcatacacactggcacagacacaagcagggacacacgcagacacacgcacactcgcgcgcgcatcctcccgccagcctgcccgcctgctcgccggcgcccggagcccgctctggccgcttgctttttgctgagaaagcttcctgccctggaagatggcacccttccccatccagacaccttgggaatgaattatgagggagccaggagtgagagagagaaccacgctgctgatgactccgagggaggggccctggacatgtgctgcagtgagaggctaccgggtctcccccagccgatagtgatggaggcactggacgaggctgaagggctccaggactcacagagagagatgccgccaccccctcctccctcgccgccctcagatccagctcagaagccaccacctcgaggcgctgggagccactccctcactgtcaggagcagcctgtgcctgttcgctgcctcacagttcctgcttgcctgtggggtgctccggttcagcggttatggccacatctggtcacagaacgccacaaacctcgtctcctctttgctgacgctcctgaaacagctggaacccacggcctggcttgactctgggacgtggggagtccccagtctgctgctggtctttctgtccgtgggcctggtcctcgttaccaccctggtgtggcacctcctgaggacacccgcagagccacccaccccactgccccctgaggacaggcgccagtcagtgagccgccagccctccttcacctactcagagtggatggaggagaagatcgaggatgacttcctggacctcgacccggtgcccgagactcctgtgtttgattgtgtgatggacatcaagcctgaggctgaccccacctcactcaccgtcaagtccatgggtctgcaggagaggaggggttccaatgtctccctgaccctggacatgtgcactccgggctgcaacgaggagggctttggctatctcatgtccccacgtgaggagtccgcccgcgagtacctgctcagcgcctcccgtgtcctccaagcagaagagcttcatgaaaaggccctggaccctttcctgctgcaggcggaattctttgaaatccccatgaactttgtggatccgaaagagtacgacatccctgggctggtgcggaagaaccggtacaaaaccatacttcccaaccctcacagcagagtgtgtctgacctcaccagaccctgacgaccctctgagttcctacatcaatgccaactacatccggggctatggtggggaggagaaggtgtacatcgccactcagggacccatcgtcagcacggtcgccgacttctggcgcatggtgtggcaggagcacacgcccatcattgtcatgatcaccaacatcgaggagatgaacgagaaatgcaccgagtattggccggaggagcaggtggcgtacgacggtgttgagatcactgtgcagaaagtcattcacacggaggattaccggctgcgactcatctccctcaagagtgggactgaggagcgaggcctgaagcattactggttcacatcctggcccgaccagaagaccccagaccgggcccccccactcctgcacctggtgcgggaggtggaggaggcagcccagcaggaggggccccactgtgcccccatcatcgtccactgcagtgcagggattgggaggaccggctgcttcattgccaccagcatctgctgccagcagctgcggcaggagggtgtagtggacatcctgaagaccacgtgccagctccgtcaggacaggggcggcatgatccagacatgcgagcagtaccagtttgtgcaccacgtcatgagcctctacgaaaagcagctgtcccaccagtccccagaatgactgcgcttctcctacaaggttctctgggcactgcccagcctgagtctcggccctcacccagggccctgcctcgggtcctgggcctgctccccgcttcctccccttcagtcagctccctctgtcctctgtcagcctggcctgacccctaccctccagcattgctcttcctactgtacatattggggagtggggggcagggtcgggaagggacatgccaggccaggcctggggccccggggcctgacccacaccacgcagaccccgggctccagtttttaacgatggttccatcaatacctgatccagaatgtttccgtgctacactttgtgtcctgctgcaatgtgttctgtctgtccatccatctctgccctctgtaccggacactgtgtctcctcagccaggaaggggtaatgagctccagcccctaagcaaccggacttgcctgcctcggcctcacccgcacttctcccaaaaggcagatgacggggagttaggcatggggagctccagaaggtcaccagagagctttcagctgagggagagttctctaggttggagtgggcatcacagccagggtggcctctgggtgtcagatgctctcaggagggtgcccagcctgtgaggcactggcaaggtagggggcagatggggcatggagaacccagaggatctaggccctgttggggaggggaggggagctcaaggtttgggtggggactcagcccagatctacgtgagacatttttctgtgtcactgtgggaaagccttcccagaagtctcactgcgtgtgttgagagcccatcaggagggcatgcatgactctttggcaacatgtattatcttggagccacgtgtttttattgctgactttaaatatttatcccacggcagacagagacatttggtgtctttttataattcgctcgtggtcattgaatagagcaataaacggagcattttgagcaaaact

SEQ ID NO: 6 - protein, 565 aa, Homo sapiens
MNYEGARSERENHAADDSEGGALDMCCSERLPGLPQPIVMEALDEAEGLQDSQREMPPPPPPSPPSDPAQKPPPRGAGSHSLTVRSSLCLFAASQFLLACGVLRFSGYGHIWSQNATNLVSSLLTLLKQLEPTAWLDSGTWGVPSLLLVFLSVGLVLVTTLVWHLLRTPAEPPTPLPPEDRRQSVSRQPSFTYSEWMEEKIEDDFLDLDPVPETPVFDCVMDIKPEADPTSLTVKSMGLQERRGSNVSLTLDMCTPGCNEEGFGYLMSPREESAREYLLSASRVLQAEELHEKALDPFLLQAEFFEIPMNFVDPKEYDIPGLVRKNRYKTILPNPHSRVCLTSPDPDDPLSSYINANYIRGYGGEEKVYIATQGPIVSTVADFWRMVWQEHTPIIVMITNIEEMNEKCTEYWPEEQVAYDGVEITVQKVIHTEDYRLRLISLKSGTEERGLKHYWFTSWPDQKTPDRAPPLLHLVREVEEAAQQEGPHCAPIIVHCSAGIGRTGCFIATSICCQQLRQEGVVDILKTTCQLRQDRGGMIQTCEQYQFVHHVMSLYEKQLSHQSPE

SEQ ID NO: 7 - DNA, 1874 bp, Mus musculus
  CDS (235)..(1275)
gactagtaaagaggctactgcggaaatttaagctacagaggagagcagtggctggaaccattctttttagtagccgcgtcctgcttctcattttcgccatgtaaaactgctgcgtgtgcgatccactctgcccccacagagcctgcagtggggtgaaatgtcaggaacaagccccggagaaggagtgagagagagagccaggctgctgatgactccgagggaggaaccctggacatgtgctgtagtgagaggctgttgggtctcccccagccggtagagatggaagcaccggacgaggccgaaggactccccagcaagcagaaagagatgccaccacccccgccaccctcaccgccctctgagccagctcagaagctgccacctcaaggcgctgggagccactccctcaccgtcagaagcagcctgtgcctgtttgctgcctctcagttcctgcttgcctgtggggtgctctggctcagtggccatggccactcctggctgcagaacaccacagacctcatctcctcctcgctcacagtgttgaaccatctgggacctgtggcctggctgggttctgggacctgggggataccaagtctgctgctagtctctctgactgtgagcctggtcatcgtcaccaccctggtgtggcacctcctcaaggcacccccagagccacctgccccactgcccccagaggacaggcgtcaatcagtgagccggcagccttccttcacctactcagagtggatggaggagaaggtagaggatgacttcctggacctggacgcggtgcccgagacacctgtgtttgactgtgtgatggacatcaagcctgagactgatcctgcctcattgactgtcaagtccatgggtctacaggagaggagaggatccaatgtctccttgaccctggacatgtgtactcctggctgcaatgaggagggcttcggctacctggtgtctccacgagaagagtcagcccatgagtatctgctcagcgcctcccgtgtcctccgggcagaagagctacatgaaaaggctctggaccctttcttgctgcaggcggaattctttgaaatccccatgaactttgtggatccaaaagagtatgacatcccagggctggtgcggaagaatcggtacaaaaccatccttcccaatcctcacagcagggtacgtctgacgtcaccagaccctgaagatcctctgagttcctacatcaatgccaactacatccgggtatgtagctccatcccaagagccttccactaaaggagaggcctgagctgatgcaatctgtccttcagacttgtcccccaagccagtgtggctcctacaagccccaagaggagcctaagtgtgggatggggccctgggagtgccacctccacccctgacctgacgggctggacttgggtggctgcagggctacagtggggaggagaaggtgtacatcgccacgcagggacccatcgtcagcactgtggccgacttttggcgcatggtgtggcaggagcgcacacccatcatcgtcatgatcaccaacatcgaggagatgaacgaggtagggaccctgtgccattgctcgttgcgccctgctctgtgcccccacatgaagcatcccctttgcataataatcttcctgaggatctcgtgagcctggcgtgcactcaactgtcaacataaggaaatgagaggtgttgacagttaaggtatacactcaacgccacacaaagcctagcagagcctggagctggcttctcccaccccatccaatggctgtatttgagtcatgatctgctgcctcccagtctgccgctgatacgcatgttcatgatgagcaagctcatttaattgaatctccattgttt

SEQ ID NO: 8 - protein, 346 aa, Mus musculus
MCCSERLLGLPQPVEMEAPDEAEGLPSKQKEMPPPPPPSPPSEPAQKLPPQGAGSHSLTVRSSLCLFAASQFLLACGVLWLSGHGHSWLQNTTDLISSSLTVLNHLGPVAWLGSGTWGIPSLLLVSLTVSLVIVTTLVWHLLKAPPEPPAPLPPEDRRQSVSRQPSFTYSEWMEEKVEDDFLDLDAVPETPVFDCVMDIKPETDPASLTVKSMGLQERRGSNVSLTLDMCTPGCNEEGFGYLVSPREESAHEYLLSASRVLRAEELHEKALDPFLLQAEFFEIPMNFVDPKEYDIPGLVRKNRYKTILPNPHSRVRLTSPDPEDPLSSYINANYIRVCSSIPRAFH

SEQ ID NO: 9 - DNA, 2198 bp, Mus sp.
  CDS (834)..(1358)
actcctggagcccgtcagtatcggcggaattccggggttgagggatggatggccttctccttctcccaaagatggctgcctggggaggttgagggatgaagacctttctgatggctccagttacttcaagtcttcatgactggagtgaagggaaggctggagttctgagctacaatctcaggaattaatttcctcatatcctgccatccctaatccctaactcaaaatcccgggtccctgagccccatgtcctacagagttctctctgaagactcctgtctaaggaggcccttctggaagacttcccttgtgtgtacctgtggggtctcctgtggggaggggctgtctcagttgcctgtgattagtttttctcctgaagagtgctgaggcctcctctgggtacaccacccttccctccactgcccagtctgagaggcttgggagcctccttgggggctgaaggtgagctctgcagggcaatataggggcaaagggctgcagccgtacactcagtctgcgcctcccaactgccccagcttgcctgtggggtgctctggctcagtggccatggccactcctggctgcagaacaccacagacctcatctcctcctcgctcacagtgttgaaccatctgggacctgtggcctggctgggttctgggacctgggggataccaagtctgctgctagtctctctgactgtgagcctggtcatcgtcaccaccctggtgtggcacctcctcaaggcacccccagagccacctgccccactgcccccagaggacaggcgtcaatcagtgagccggcagccttccttcacctactcagagtggatggaggagaaggtagaggatgacttcctggacctggacgcggtgcccgagacacctgtgtttgactgtgtgatggacatcaagcctgagactgatcctgcctcattgactgtcaagtccatgggtctacaggagaggagaggatccaatgtctccttgaccctggacatgtgtactcctggctgcaatgaggagggcttcggctacctggtgtctccacgagaagagtcagcccatgagtatctgctcagcgcctcccgtgtcctccgggcagaagagctacatgaaaaggctctggaccctttcttgctgcaggcggaattctttgaaatccccatgaactttgtggatccaaaagagtatgacatcccagggctggtgcggaagaatcggtacaaaaccatccttcccaatcctcacagcagggtacgtctgacgtcaccagaccctgaagatcctctgagttcctacatcaatgccaactacatccgggtatgtagctccatcccaagagccttccactaaaggagaggcctgagctgatgcaatctgtccttcagacttgtcccccaagccagtgtggctcctacaagccccaagaggagcctaagtgtgggatggggccctgggagtgccacctccacccctgacctgacgggctggacttgggtggctgcagggctacagtggggaggagaaggtgtacatcgccacgcagggacccatcgtcagcactgtggccgacttttggcgcatggtgtggcaggagcgcacacccatcatcgtcatgatcaccaacatcgaggagatgaacgagaagtgcacagaatactggccagaagagcaggtggtccatgacggcgtggagatcaccgtgcagaaagtcatccacacggaggactaccggctgcgactcatctccctcagggtgaggcatggacctaaacaggaaagggatgttggggaggcttagttgagacccccacctgaaaacagtactgaaaggcctactgtaggtacacatcgacccacggcactcccagcacgtactgttaaacaagggtcaatagactcagacgctaagccttgcttgcatgtgccctacgccctttctgtgcactgccgaccatagcagctctcctccacatcttcccaaggccagcactacctgggcaaggaggcttcaagggcttccagcattgtccttatcgtaactgagagttcctttacgagtttgagtgacagtaatgtcccttccactggggtgctggcttccagtctgatgcacccaaaatgtccaccagcagagggcaggcactccttgctgctttaggacacacagtcatggttacaggaaatggcagg

SEQ ID NO: 10 - protein, 174 aa, Mus sp.
MEEKVEDDFLDLDAVPETPVFDCVMDIKPETDPASLTVKSMGLQERRGSNVSLTLDMCTPGCNEEGFGYLVSPREESAHEYLLSASRVLRAEELHEKALDPFLLQAEFFEIPMNFVDPKEYDIPGLVRKNRYKTILPNPHSRVRLTSPDPEDPLSSYINANYIRVCSSIPRAFH

SEQ ID NO: 11 - DNA, 1635 bp, Rattus norvegicus
  CDS (13)..(1632)
gcttctagaaccatgtgctgtagtgagaggctgttgggtcgcccccagccagtagagatgggaggtctggacgagggcgaaggactccccagcaagcagagagagctgccacccccgccaccctcaccgccctctgagccagctcagaagctgccacctcarggtgccgggagccactccctcaccgtcagaagcagcctgtgcctgtttgctgcctctcagttactgcttgcctgtggggtgctctggctcagtggccatggccactcctggctgcagaccaccacagacctcatcttctcctcgctcaccgtgttgaaccacctgggacctgcagcctggctaggttctgggacctgggggacaccaagtctgctgttagtctctctgactgtgagcctgggcatcatcaccaccctggtgtggcaccttctcaagacacccccagagccacctgtcccactgcccccagaggacagacgacaatcagtgagccggcagccctccttcacttactcagagtggatggaggagaaggtagaggatgactttctggacctggacgcagtgccggagacacccgtgtttgactgtgtgatggacatcaagcctgaggctgatcccacctcgttgactgtcaagtccatgggtctgcaggagaggaggggctccaatgtctccttgaccctggacatgtgtactcctggctgcaatgaggagggctttggctacctggtgtccccacgagaggagtcagcccatgaatacctgctcagtgcctcccgtgtcctccgggcagaggagctgcacgaaaaggccctggaccctttcttgctgcaggcggaattctttgaaatccccatgaactttgtggatccaaaagaatatgacatcccagggctggtacggaagaatcggtacaaaaccatccttcccaatcctcacagcagggtacgtctgacgtcaccagaccctgaagatcctctgagttcctacatcaatgccaactatatccggggctacaatggggaggagaaggtgtacatcgccacccagggacccatcgtcagcactgtggtcgacttctggcgcatggtgtggcaggagcgcacacccatcatcgtcatgatcaccaacatcgaggagatgaacgagaagtgcacggaatattggccagaagagcaggtcgtccacgatggcgtagagatcactgtgcagaaagtcatccacacagaggactaccggctgcgacttatctccctcaggagagggactgaagagagaggcttgaagcattactggttcacatcctggcctgaccagaagacccccgaccgggcacccccactcctgcacctggtacgggaggtggaggaggcagcccagcaagagggaccccactgttccccgatcattgttcactgcagtgcagggattgggagaaccgggtgcttcatcgccaccagcatctgctgccagcagctgcgacgtgagggcgtggtagacatcctaaagaccacgtgccagctccgtcaggacaggggcggcatgatccaaacatgcgaacagtatcagtttgtgcaccacgccatgagcctctatgagaagcagctatccctccagtcctcagagtga

SEQ ID NO: 12 - protein, 540 aa, Rattus norvegicus
MCCSERLLGRPQPVEMGGLDEGEGLPSKQRELPPPPPSPPSEPAQKLPPQGAGSHSLTVRSSLCLFAASQLLLACGVLWLSGHGHSWLQTTTDLIFSSLTVLNHLGPAAWLGSGTWGTPSLLLVSLTVSLGIITTLVWHLLKTPPEPPVPLPPEDRRQSVSRQPSFTYSEWMEEKVEDDFLDLDAVPETPVFDCVMDIKPEADPTSLTVKSMGLQERRGSNVSLTLDMCTPGCNEEGFGYLVSPREESAHEYLLSASRVLRAEELHEKALDPFLLQAEFFEIPMNFVDPKEYDIPGLVRKNRYKTILPNPHSRVRLTSPDPEDPLSSYINANYIRGYNGEEKVYIATQGPIVSTVVDFWRMVWQERTPIIVMITNIEEMNEKCTEYWPEEQVVHDGVEITVQKVIHTEDYRLRLISLRRGTEERGLKHYWFTSWPDQKTPDRAPPLLHLVREVEEAAQQEGPHCSPIIVHCSAGIGRTGCFIATSICCQQLRREGVVDILKTTCQLRQDRGGMIQTCEQYQFVHHAMSLYEKQLSLQSSE

(19) United States
(12) Patent Application Publication
(10) Pub. No.: US 2005/0221411 A1
(43) Pub. Date: Oct. 6, 2005

(54) Interaction of NMDA receptor with the protein tyrosine phosphatase step in psychotic disorders

(75) Inventors: Steven Braithwaite, San Francisco, CA (US); Jasna Jerecic, San Francisco, CA (US); Roman Urfer, Belmont, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: AGY Therapeutics, Inc.

(21) Appl. No.: 11/107,597

(22) Filed: Apr. 15, 2005

Related U.S. Application Data
(63) Continuation-in-part of application No. 11/008,354, filed on Dec. 8, 2004.
(60) Provisional application No. 60/528,182, filed on Dec. 8, 2003.

(51) Int. Cl. C12Q 1/48; C12Q 1/42

(52) U.S. Cl. 435/15; 435/21

(57) ABSTRACT

The present invention relates to the identification of STEP being as involved in signaling pathways relating to psychotic diseases, including schizophrenia, and other disorders in which NMDA receptor dysfunction is implicated. The present invention provides methods for screening STEP inhibitors that modulate NMDA-R signaling. The present invention also provides methods and compositions for treatment of disorders mediated by abnormal NMDA-R signaling.

### BACKGROUND OF THE INVENTION

In the majority of mammalian excitatory synapses, glutamate (Glu) mediates rapid chemical neurotransmission by binding to four distinct types of glutamate receptors on the surfaces of brain neurons. Although cellular responses mediated by glutamate receptors are normally triggered by exactly the same excitatory amino acid (EAA) neurotransmitters in the brain (e.g., glutamate or aspartate), the different subtypes of glutamate receptors have different patterns of distribution in the brain, and mediate different cellular signal transduction events. One major class of glutamate receptors is referred to as N-methyl-D-aspartate receptors (NMDA-Rs), since they bind preferentially to N-methyl-D-aspartate (NMDA). NMDA is a chemical analog of aspartic acid; it normally does not occur in nature, and NMDA is not present in the brain. When molecules of NMDA contact neurons having NMDA-Rs, they strongly activate the NMDA-R (i.e., they act as a powerful receptor agonist), causing the same type of neuronal excitation that glutamate does. It has been known that excessive activation of NMDA-R plays a major role in a number of important central nervous system (CNS) disorders, while hypoactivity of NMDA-R has been implicated in several psychiatric diseases.

NMDA-Rs contain NR1 or NR3 subunits and at least one of four different NR2 subunits (designated as NR2A, NR2B, NR2C, and NR2D). NMDA-Rs are “ionotropic” receptors since they flux ions, such as Ca2+. These ion channels allow ions to flow into a neuron upon depolarization of the postsynaptic membrane , when the receptor is activated by glutamate, aspartate, or an agonist drug.

Protein tyrosine phosphorylation plays an important role in regulating diverse cellular processes. The regulation of protein tyrosine phosphorylation is mediated by the reciprocal actions of protein tyrosine kinases (PTKs) and protein tyrosine phosphatases (PTPs). NMDA-Rs are regulated by protein tyrosine kinases and phosphatases. Phosphorylation of NMDA-R by protein tyrosine kinases results in enhanced NMDA-R responsiveness in neurons (Wang et al., Nature 369:233-235, 1994). NR2B and NR2A have been shown to be the main sites of phosphorylation by protein tyrosine kinases. Protein tyrosine phosphatases, on the other hand, exert opposing effects on the responsiveness of NMDA-R in the neurons (Wang et al, Proc. Natl. Acad. Sci. U.S.A. U.S.A. 93:1721-1725, 1996). It is believed that members of the Src family of protein tyrosine kinases mediate the NMDA-R tyrosine phosphorylation. On the other hand, the identity of the enzyme responsible for the counter dephosphorylation of NMDA-R has been elusive.

Most psychiatric disorders are classified as complex in origin, arising from interactions between genetic and environmental causes. One of the most debilitating of these disorders is schizophrenia, which affects about 1% of the population. Once the symptoms occur, usually in young adulthood, they persist for the entire lifetime of the patient and are almost totally disabling. Diagnosis is based on the simultaneous presentation of two types of symptoms that reflect a psychotic disturbance: “positive” symptoms that include delusions, hallucinations, and bizarre thoughts, and negative symptoms that include social withdrawal with affective flattening, poor motivation, and apathy.

Although the clinical efficacy of dopamine D2 receptor blockers suggests a dopamine imbalance is important in schizophrenia, it has become clear that several other neurotransmitter systems, including the glutamatergic system, are also involved in the pathophysiology of the schizophrenic brain. Positive modulators of cortical glutamatergic systems may be useful adjuncts in treating schizophrenia.

Glutamatergic transmission is known, to play a fundamental role in cognitive processes. Accumulating evidence suggests that reduced excitatory (glutamatergic) activity, especially involving select neocortical areas, could underlie some, if not many, symptoms of schizophrenia. For example, see Coyle (1996) Harv Rev Psychiatry 3:241-253; and Tamminga (1998) Crit Rev Neurobiol 12:21-36. Imaging and postmortem morphometry studies of schizophrenic brains have found abnormalities in a number of brain regions, such as prefrontal, temporal and anterior cingulated cortices, hippocampus, amygdala, and striatum, that are connected by glutamatergic circuits. Phencyclidine, ketamine, and other noncompetitive antagonists at N-methyl-D-aspartate (NMDA)-type glutamate receptors exacerbate symptoms in patients (Lahti et al. (1995) Neuropsychopharmacology 13:9-19) and produce a range of psychotic symptoms in volunteers that are similar to those of schizophrenic patients.

Drugs that enhance glutamatergic transmission might offset the postulated imbalance between ascending midbrain monoaminergic systems and descending cortical glutamatergic systems in the schizophrenic brain (Carlsson and Carlsson (1990) Trends Neurosci. 13:272-276). One approach has centered on enhancing NMDA receptor activity with glycine or related agonists (D-cycloserine) of the strychnine-insensitive glycine coagonist site. Some beneficial effects of D-cycloserine on negative symptoms in patients coadministered a typical antipsychotic have been reported. Methods of screening active compounds, and the use of such compounds in treating schizophrenia have substantial medical interest.

### SUMMARY OF THE INVENTION

Methods are provided for identifying agents therapeutic in the treatment of psychotic disorders, including schizophrenia and related conditions, by screening for inhibitors of N-methyl-D-aspartate receptor (NMDA-R) signaling that act through one or more isoforms of the protein tyrosine phosphatase STEP. In one embodiment, the modulator is identified by detecting its ability to modulate the phosphatase activity of STEP. In another embodiment, the modulator is identified by detecting its ability to modulate the binding of STEP and the NMDA-R. In another embodiment, methods are provided for identifying a nucleic acid molecule encoding polypeptides that modulate NMDA-R signaling. It is found that active STEP downregulates NMDA-R activity, and inhibitors of STEP can increase the activity of NMDA-R when STEP is present.

Methods are provided for treating schizophrenia and related disorders by administering an inhibitor of STEP activity, which directly or indirectly modulates the tyrosine phosphorylation level of the NMDA-R. The modulator may affect the ability of STEP to dephosphorylate NMDA-R, to dephosphorylate kinases, e.g. ERK, in a signaling pathway associated with NMDA-R, and/or the ability of STEP to bind to NMDA-R. In certain embodiments, the modulator is a STEP antagonist and the disease to be treated is mediated by NMDA-R hypofunction.

### BRIEF DESCRIPTION OF THE DRAWINGS

FIG. 1. STEP is expressed selectively in brain as detected by quantitative PCR in multiple rat tissues. Quantitative PCR using probes that recognize both STEP46 and STEP61 isoforms (top) and STEP61 alone (bottom) show that STEP mRNA is specifically localized in the brain.

FIG. 2. STEP is expressed selectively in brain as detected by Northern blot in multiple rat tissues.

FIG. 3. STEP is expressed selectively in brain as detected by Northern blot in multiple human tissues.

FIG. 4A-B. STEP is expressed in rat brain as shown by in situ hybridization high levels in striatum and hippocampus. In situ hybridization of rat brain sections with probes to STEP show strong expression in striatum, CA2 and subiculum and detectable expression in other hippocampal regions and in cortex.

FIG. 5. Overexpression of STEP causes decreased NMDA receptor function. HEK293 cells stably expressing NR1 and NR2B subunits were transfected with constructs of STEP61 (61 (WT)), STEP46 (46(WT)) or forms of either which contain a C-S mutation in their catalytic domains which makes them inactive (61 (CS) and 46(CS)). Cells were loaded with a calcium indicator dye and the Ca influx into cells elicited by application of 1 μM glutamate was measured by assessing the fluorescence change. The mean response to glutamate was normalized to the total cell number by assessing the fluorescence change elicited by permeabilization of cells with 1% NP40.

FIG. 6. Knockdown of STEP levels causes an increase in NMDA receptor function. Cultured cortical neurons were transfected with inhibitory RNA molecules designed to specifically inhibit STEP expression using the Amaxa Nucleofection technique. (Top) Four days after transfection Western blot analysis shows that neurons transfected with inhibitory RNA to STEP show lower levels of STEP61 protein than those transfected with a scrambled RNA molecule. (Bottom) Measurement of the Ca influx elicited by application of 1 μM NMDA to neurons four days after transfection shows a larger NMDA response in cells whose levels of STEP61 expression have been reduced than those in which a scrambled RNA molecule was introduced.

FIG. 7. STEP causes decreased ERK phosphorylation in transfected HEK-293 cells. STEP46 causes a decrease in EGF stimulated ERK phosphorylation in transfected HEK293 cells. HEK293 cells were transfected with various constructs, 2 days after transfection cells were treated with 50 ng/ml EGF for 15 mins. Cells were lysed and proteins separated by SDS-polyacrylamide gel electrophoresis. Proteins were transferred to nitrocellulose membranes and these were probed with antibodies that specifically recognize phosphorylated ERK. In the presence of an active form of STEP46 (46WT) ERK phosphorylation is reduced compared to untransfected cells. A catalytically inactive form of STEP46 (46CS) shows much increased phosphorylation. PTP-MEG expression either in active (MEG WT) or inactive (MEG CS) has no effect on ERK phosphorylation.

FIG. 8A-B. STEP modulates NMDAR mediated ERK phosphorylation in neurons. Cultured cortical neurons (10-13 division) show low levels of basal ERK phosphorylation. Upon addition of 100 μM NMDA for 5 minutes ERK phosphorylation levels are significantly increased. Application of the NMDA receptor antagonist D-APV (200 μM) inhibits NMDA. stimulated ERK phosphorylation (left panel). Infection of neurons with sindbis virus containing RNA encoding GFP, STEP61, or STEP 61cs shows that STEP affects NMDAR mediated ERK phosphorylation. One day after infection of cultured cortical neurons with sindbis virus cells were treated with 100 μM glutamate for 5 minutes and harvested. SDS-PAGE was performed and western blotting used to detect ERK phosphorylation levels. Neurons infected with active STEP show less ERK phosphorylation than GFP (control) infected cells. Neurons infected with the dominant negative STEPcs show more phosphorylation of ERK than GFP infected cells (right panel).

FIG. 9A-B. HEK293 cells transfected with STEP61 and Fyn (top) or Src (bottom) show a concentration dependent decrease in the phosphorylation state of the kinase. Cells were transfected with constitutively active forms of either kinase and varying amounts of STEP61. Two days after transfection cells were lysed and proteins separated by SDS-polyacrylamide gel electrophoresis. Proteins were transferred to nitrocellulose membranes and these were probed with antibodies that specifically recognize phosphorylated forms of the kinase (Src-PY-418 or Fyn-PY-420). With increasing amounts of STEP61 levels of phosphorylation at these sites are decreased.

FIG. 10. HEK293 cells stably transfected with NR1, NR2B and STEP61 were harvested. Immunoprecipitation was performed with anti-NR1 antibody (left panel) or anti-STEP (right panel). Lysates were incubated overnight with antibodies, protein G sepharose was then added to each lysate for 1 hour and then immunoprecipitated proteins isolated by SDS-PAGE. Western blotting shows that STEP and NR1 containing NMDAR co-immunoprecipitate in stably expressing cell lines.

FIG. 11. STEP61 interacts with NR1 and NR2 subunits of NMDAR. HEK293 cells were transfected with NR1, NR2A or NR2B and STEP61. Immunoprecipitation was preformed with appropriated subunit selective antibodies. Left panels show that co-immunoprecipitation of STEP61 with NMDAR subunits occurs when complexes are pulled down with antibodies to specific subunits. Right panels show that individual NMDAR subunits are co-immunoprecipitated with STEP61 when complexes are pulled down with anti-STEP antibody.

FIG. 12. STEP46 interacts with NR1 and NR2 subunits of NMDAR. HEK293 cells were transfected with NR1, NR2A or NR2B and STEP61. Immunoprecipitation was preformed with appropriated subunit selective antibodies. Left panels show that co-immunoprecipitation of STEP46 with NMDAR subunits occurs when complexes are pulled down with antibodies to specific subunits. Right panels show that individual NMDAR subunits are co-immunoprecipitated with STEP46 when complexes are pulled down with anti-STEP antibody.

FIG. 13. Surface expression of NMDA receptors is specifically increased by knockdown of STEP levels by RNA interference. RNAi-STEP or RNAi-Scrambled were introduced into neurons by AMAXA nucleofection. Surface expressed receptors were labeled with biotin and cells harvested. Biotinylated proteins were isolated by pulldown with neutravadin agarose and separated by SDS-PAGE. Western blots were performed with subunit selective antibodies. In all experiments it was confirmed that STEP had been knocked down with RNAi-STEP relative to RNAi-Scrambled by assessing STEP levels in the total protein lysate. Levels of surface NR1, NR2A and NR2B were all increased in neurons in which RNAi-STEP had been introduced. Surface levels of EGF receptors, the GluR1 subunit of AMPA receptors and the GABAA receptor b subunit were unaffected by RNAi-STEP. Total levels of each subunit were the same in neurons electroporated with RNAi-STEP or RNAi-Scrambled. Images are representative of at least 3 experiments.

### DETAILED DESCRIPTION OF THE EMBODIMENTS

The present invention relates to modulation of the binding interaction between the NR2A or NR2B subunits of the NMDA-R and STEP protein tyrosine phosphatase. In accordance with the discovery, the present invention provides methods for identifying agonists and antagonists of STEP that modulate NMDA-R signaling, and for treating conditions mediated by abnormal NMDA-R signaling. Of particular interest is the treatment of schizophrenia. The following description provides guidance for making and using the compositions of the invention, and for carrying out the methods of the invention.

In culture models, downstream signaling events in the NMDA-R signaling pathway are affected by STEP expression, where overexpression of STEP causes a decrease in either EGF or glutamate stimulated ERK phosphorylation. Phosphorylated ERK is a key signaling molecule between NMDA receptor activation and nuclear events, as it in turn affects CREB phosphorylation and genes whose transcription is under the regulation of CREB. Thus the downstream signaling mediated by NMDA-Rs is affected by STEP, and STEP exacerbates the effects of reduced NMDA-R function in schizophrenia.

STEP causes decreased phosphorylation of the tyrosine kinases fyn and src, when it is overexpressed in HEK293 cells. Both src and fyn are known to phosphorylate NMDA receptors when they are in active, phosphorylated forms, so STEP acts to decrease the phosphorylation level of NMDA-R. Less phosphorylated NMDA-Rs have lower conductance states and so will allow less current and fewer ions to pass and so will be functionally less active. This can lead to schizophrenic symptoms.

### DEFINITIONS

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). Although any methods and materials similar or equivalent to those described herein can be used in the practice or testing of the present invention, the preferred methods and materials are described. The following definitions are provided to assist the reader in the practice of the invention.

As used herein, the term “psychotic disorder” has the meaning as commonly known in the art, and as set forth in the Diagnostic and Statistical Manual of Mental Disorders, Fourth Edition. Among the symptoms of psychotic disorders are delusions, hallucinations, disorganized speech (e.g., frequent derailment or incoherence), and grossly disorganized or catatonic behavior.

Schizophrenia is a common and serious mental disorder. In the USA, patients with schizophrenia occupy about ¼ of all hospital beds and account for about 20% of all social security disability days. Schizophrenia is more prevalent than Alzheimer's disease, diabetes, or multiple sclerosis. Symptoms of schizophrenia vary in type and severity. Generally they are categorized as positive or negative (deficit) symptoms. Positive symptoms can be further categorized as delusions and hallucinations; or thought disorder and bizarre behavior. Delusions and hallucinations are sometimes referred to as the psychotic dimension of schizophrenia. Thought disorder and bizarre behavior are termed the disorganized symptom cluster. Negative (deficit) symptoms include blunted affect, poverty of speech, anhedonia, and asociality. In some patients with schizophrenia, cognitive functioning declines, with impaired attention, abstract thinking, and problem solving. Severity of cognitive impairment is a major determinant of overall disability in these patients.

Although its specific cause is unknown, schizophrenia has a biologic basis. A vulnerability-stress model, in which schizophrenia is viewed as occurring in persons with neurologically based vulnerabilities, is the most widely accepted explanation. Onset, remission, and recurrence of symptoms are seen as products of interaction between these vulnerabilities and environmental stressors. Although many clinical and experimental vulnerability markers have been proposed, none is ubiquitous. Psychophysiologically, deficits in information processing, attention, and sensory inhibition may be markers for vulnerability. Although most persons with schizophrenia do not have a family history of it, genetic factors have been implicated. Persons who have a first-degree relative with schizophrenia have about a 15% risk of developing the disorder, compared with a 1% risk among the general population. A monozygotic twin whose co-twin has schizophrenia has a >50% probability of developing it.

Conventional antipsychotic (neuroleptic) drugs include chlorpromazine, fluphenazine, haloperidol, loxapine, mesoridazine, molindone, perphenazine, pimozide, thioridazine, thiothixene, and trifluoperazine. These drugs are characterized by their affinity for the dopamine 2 receptor and can be classified as high, intermediate, or low potency. Atypical antipsychotic drugs may have selective affinity for brain regions involved in schizophrenia symptoms and reduced affinity for areas associated with motor symptoms and prolactin elevation. They affect other neurotransmitter systems, including serotonin, or have selective affinity for specific dopamine receptor subtypes.

The aberrant behaviors induced in rats by methamphetamine (Larson et al. (1996) Brain Res 738:353-356), is a common and often predictive test of antipsychotic drug activity. Implicit in the hypothesis that schizophrenia arises from an imbalance between opposing neurotransmitter systems is the prediction that antagonists of one of the systems and positive modulators of the other should be at least additive and probably synergistic. This is of considerable clinical significance because it suggests a novel therapeutic strategy involving low levels of two completely different classes of drugs. Reducing the dose of commonly used antipsychotics should reduce their often treatment-limiting side effects.

Psychotic disorders other than schizophrenia include schizophreniform disorder, which is diagnosed when the symptom criteria for Schizophrenia are met, but the duration is too short and social and occupational functioning may not be impaired. In schizoaffective disorder, the symptom criteria for Schizophrenia are met, and during the same continuous period there is a major depressive, manic or mixed episode. With delusional disorder, prominent nonbizarre delusions are present for at least one month and the symptom criteria for schizophrenia have never been met. Brief psychotic disorder is diagnosed when psychotic symptoms such as delusions, hallucinations, or disorganized or catatonic speech or behavior are present for less than a month and resolve completely. Shared psychotic disorder is diagnosed when delusions develop in an individual involved in a close relationship with another individual already afflicted with delusions arising out of a different psychosis.

Psychotic conditions can also arise from other illnesses, or from substance abuse. Associated with these disorders are: alcohol, amphetamine-like, cannabis, cocaine, hallucinogens, inhalants, opioids, phencyclidine, sedatives, and hypnotics.

The term “agent” includes any substance, molecule, element, compound, entity, or a combination thereof. It includes, but is not limited to, protein, oligopeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

As used herein, an “agonist” is a molecule which, when interacting with (e.g., binding to) a target protein (e.g., STEP, NMDA-R), increases or prolongs the amount or duration of the effect of the biological activity of the target protein. By contrast, the term “antagonist,” as used herein, refers to a molecule which, when interacting with (e.g., binding to) a target protein, decreases the amount or the duration of the effect of the biological activity of the target protein (e.g., STEP or NMDA-R). Agonists and antagonists may include proteins, nucleic acids, carbohydrates, antibodies, or any other molecules that decrease the effect of a protein. Unless otherwise specified, the term “agonist” can be used interchangeably with “activator”, and the term “antagonist” can be used interchangeably with “inhibitor”.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

The term “biological preparation” refers to biological samples taken in vivo and in vitro (either with or without subsequent manipulation), as well as those prepared synthetically. Representative examples of biological preparations include cells, tissues, solutions and bodily fluids, a lysate of natural or recombinant cells.

As used herein, the term “functional derivative” of a native protein or a polypeptide is used to define biologically active amino acid sequence variants that possess the biological activities (either functional or structural) that are substantially similar to those of the reference protein or polypeptide. Thus, a functional derivative of a PTP may retain, among other activities, the ability to bind to, and dephosphorylate NMDA-R. Similarly, a functional derivative of NMDA-R may be capable of binding to a PTP, and of being dephosphorylated by a PTP.

NMDA receptors are a subclass of excitatory, ionotropic L-glutamate neurotransmitter receptors. They are heteromeric, integral membrane proteins being formed by the assembly of the obligatory NR1 subunit together with modulatory NR2 subunits. The NRI subunit is the glycine binding subunit and exists as 8 splice variants of a single gene. The glutamate binding subunit is the NR2 subunit, which is generated as the product of four distinct genes, and provides most of the structural basis for heterogeneity in NMDA receptors. In the hippocampus and cerebral cortex, the active subunit NMDAR1 is associated with 1 of 2 regulatory epsilon subunits: NMDAR2A or NMDAR2B and NR3. Unless otherwise specified, the term “NMDA-R” or “NMDA receptor” as used herein refers to an NMDA receptor molecule that has an NR1 subunit and at least one NR2A or NR2B subunit.

An exemplary NR1 subunit is the human NMDA-R1 polypeptide. The sequence of the polypeptide and corresponding nucleic acid may be obtained at Genbank, accession number L05666, and is published in Planells-Cases et al. (1993) P.N.A.S. 90(11):5057-5061. An exemplary NR2 subunit is the human NMDAR2A polypeptide. The sequence of the polypeptide and corresponding nucleic acid may be obtained at Genbank, accession number U09002, and is published in Foldes et al. (1994) Biochim. Biophys. Acta 1223 (1):155-159. Another NR2 subunit is the human NMDAR2B polypeptide. The sequence of the polypeptide and corresponding nucleic acid may be obtained at Genbank, accession number U11287, and is published in Adams et al. (1995) Biochim. Biophys. Acta 1260 (1):105-108.

The protein tyrosine phosphatase STEP is characterized by an association with NMDA-R in vivo, particular in neural tissue, more particularly in brain tissue. A fundamental process for regulating the function of NMDA receptors and other ion channels in neurons is tyrosine phosphorylation. A phosphatase enzyme may act on NMDA-R directly, to dephosphorylate one or more of the NMDA-R subunits. Alternatively a phosphatase enzyme may act on NMDA-R indirectly, by dephosphorylating a protein tyrosine kinase (PTK) in a signaling pathway. For example, a phosphatase that acts to decrease the activity of a PTK that phosphorylates NMDA-R, will indirectly result in decreased phosphorylation of NMDA-R.

The protein tyrosine phosphatase STEP is also referred to as PTPN5. In the brain, there are STEP transcripts of 3 kb, which is highly enriched in the striatum relative to other areas, termed striatum-enriched phosphatase (STEP); and a 4.4-kb mRNA, which is most abundant in the cerebral cortex and rare in the striatum. See Genomics (1995). 28(3):442-9; and Proc Natl Acad Sci USA (1991) 88(16):7242-6.

Among the transcripts of STEP are 6 different transcripts, altogether encoding 6 different protein isoforms. There are 4 probable alternative promotors and 2 non overlapping alternative last exons. The transcripts appear to differ by truncation of the N-terminus, truncation of the C-terminus, presence or absence of 2 cassette exons, common exons with different boundaries. The tyrosine specific protein phosphatase motif is found in 3 isoforms from this gene. Among the STEP isoforms are STEP 46, which is the full-length, 46-kD protein and is cytoplasmic. STEP 20 lacks the tyrosine phosphatase domain. STEP 61 has a 5-prime extended open reading frame that encodes a protein with a predicted molecular mass of 61 kD , contains a single tyrosine phosphatase domain and is membrane bound. The sequences may be accessed as Genbank: NM_{—}032781; AL832541; AK055450; and BI668912.

It has been shown that glutamate-mediated activation of N-methyl-D-aspartate (NMDA) receptors leads to the rapid but transient phosphorylation of extracellular signal-related kinase (ERK; MAPK1) (Paul et al. (2003) Nature Neurosci. 6:34-42). NMDA-mediated influx of calcium led to activation of calcineurin and the subsequent dephosphorylation and activation of STEP. STEP then inactivated ERK through dephosphorylation of the tyrosine residue in its activation domain and blocked nuclear translocation of the kinase. Thus, STEP is important in regulating the duration of ERK activation and downstream signaling in neurons.

Sequences of exemplary STEP polypeptides and nucleic acids may be found as set forth in Table 1, and in the attached Seqlist.

| | | NT | SEQ | PROTEIN | SEQ | RELATED |
|---|---|---|---|---|---|---|
| AGY ID | DESCRIPTION | ACCESSION | ID | ACCESSION | ID | ACCESSIONS |
| PL00188_G05 | AGY Homo | N/A | 1 | N/A | 2 | N/A |
| | sapiens STEP61 | | | | | |
| | full-length clone | | | | | |
| PL00188_G05 | Human (STEP) | U27831 | 3 | AAA87555 | 4 | N/A |
| | mRNA, | | | | | |
| PL00188_G05 | Homo sapiens | NM_032781 | 5 | NP_116170 | 6 | AK090923 |
| | mRNA | | | | | AK055450 |
| | | | | | | AK127312 |
| | | | | | | AK027333 |
| | | | | | | AL832541 |
| | | | | | | B1668912 |
| PL00188_G05 | Mus musculus | U28216 | 7 | AAA73573 | 8 | AK038146 |
| | STEP38 mRNA, | | | | | NM_013643 |
| PL00188_G05 | STEP20 mRNA | S80329 | 9 | AAB35656 | 10 | AK038146 |
| | | | | | | NM_013643 |
| PL00188_G05 | AGY Rattus | N/A | 11 | N/A | 12 | S49400 |
| | norvegicus | | | | | NM_019253 |
| | STEP61 full-length | | | | | |
| | clone | | | | | |

Protein kinases have been found to potentiate the function of recombinant NMDA receptors, including the mitogen-activated protein (MAP) kinase group, or ERKs. MAPK1 is also known as ERK, or p42MAPK. The MAP kinase ERK is widely involved in eukaryotic signal transduction. Upon activation, it translocates to the nucleus of the stimulated cell, where it phosphorylates nuclear targets. Nuclear accumulation of microinjected ERK depends on its phosphorylation state rather than on its activity or on upstream components of its signaling pathway. Phosphorylated ERK forms dimers with phosphorylated and unphosphorylated ERK partners. Disruption of dimerization by mutagenesis of ERK reduces its ability to accumulate in the nucleus, suggesting that dimerization is essential for its normal ligand-dependent relocalization. Other MAP kinase family members also form dimers. For a review, see Bhalla et al. (2002) Science 297:1018-1023. The sequence of ERK may be accessed at Genbank, accession number M84489; and is described by Owaki et al. (1992) Biochem. Biophys. Res. Commun. 182 (3), 1416-142.

Other protein kinases associated with NMDA-R signaling include the family of Src kinases, which comprises a total of nine members. Five members of this family: Src, Fyn, Lyn, Lck, and Yes, are known to be expressed in the CNS. All members of the Src family contain highly homologous regions the C-terminal, catalytic, Src homology 2, and Src homology 3 domains. The kinase activity of Src protein is normally inactivated by phosphorylation of the tyrosine residue at position 527, which is six residues from the C-terminus. Hydrolysis of phosphotyrosine 527 by a phosphatase enzyme normally activates c-Src.

As used herein, the term “NMDA-R signaling” refers to signal-transducing activities in the central nervous system that are involved in the various cellular processes such as neurodevelopment, neuroplasticity, and excitotoxicity. NMDA-R signaling affects a variety of processes including, but not limited to, neuron migration, neuron survival, synaptic maturation, learning and memory, and neurodegeneration.

The term “NMDA-R hypofunction” is used herein to refer to abnormally low levels of signaling activity of NMDA-Rs on CNS neurons. For example, NMDA-R hypofunction may be caused by abnormally low phosphotyrosine level of NMDA-R. NMDA-R hypofunction can occur as a drug-induced phenomenon. It can also occur as an endogenous disease process, and is associated with schizophrenia and psychotic disorders.

The term “modulation” as used herein refers to both upregulation, (i.e., activation or stimulation), for example by agonizing; and downregulation (i.e. inhibition or suppression), for example by antagonizing, of a bioactivity (e.g., direct or indiriect NMDA-R tyrosine phosphorylation, STEP tyrosine phosphatase activity, STEP binding to NMDA-R). As used herein, the term “modulator of NMDA-R signaling” refers to an agent that is able to alter an NMDA-R activity that is involved in the NMDA-R signaling pathways. Modulators include, but are not limited to, both “activators” and “inhibitors” of NMDA-R tyrosine phosphorylation. An “activator” is a substance that directly or indirectly enhances the tyrosine phosphorylation level of NMDA-R, and thereby causes the NMDA receptor to become more active. The mode of action of the activator may be direct, e.g., through binding the receptor, or indirect, e.g., through binding another molecule which otherwise interacts with NMDA-R (e.g., STEP, Src, Fyn, ERK, etc). Conversely, an “inhibitor” directly or indirectly decreases the tyrosine phosphorylation of NMDA-R, and thereby causes NMDA receptor to become less active. The reduction may be complete or partial. As used herein, modulators of NMDA-R signaling encompass STEP antagonists and agonists.

As used herein, the term “PTP modulator” includes both “activators” and “inhibitors” of PTP phosphatase activity. An “activator” of PTP is a substance that causes a PTP to become more active, and thereby directly or indirectly decreases the phosphotyrosine level of NMDA-R. The mode of action of the activator may be through binding the PTP; through binding another molecule which otherwise interacts with the PTP; etc. Conversely, an “inhibitor” of a PTP is a substance that causes the PTP to become less active, and thereby directly or indirectly increases phosphotyrosine level of NMDA-R. The reduction may be complete or partial, and due to a direct or an indirect effect.

As used herein, the term “STEP/NMDA-R-containing protein complex” refers to protein complexes, formed in vitro or in vivo, that contain STEP and NMDA-R. In addition, the complex may also comprise other components, e.g., a protein tyrosine kinase such as Fyn,. Src, etc.

The terms “substantially pure” or “isolated,” when referring to proteins and polypeptides, e.g., a fragment of a PTP, denote those polypeptides that are separated from proteins or other contaminants with which they are naturally associated. A protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition.

A “variant” of a molecule such as STEP or NMDA-R is meant to refer to a molecule substantially similar in structure and biological activity to either the entire molecule, or to a fragment thereof. Thus, provided that two molecules possess a similar activity, they are considered variants as that term is used herein if the composition or secondary, tertiary, or quaternary structure of one of the molecules is not identical to that found in the other, or if the sequence of amino acid residues is not identical.

As used herein, “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide.

The term “operably linked” refers to functional linkage between a nucleic acid expression control sequence (such as a promoter, signal sequence, or array of transcription factor binding sites) and a second polynucleotide, wherein the expression control sequence affects transcription and/or translation of the second polynucleotide.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “heterologous sequence” or a “heterologous nucleic acid,” as used herein, is one that originates from a source foreign to the particular host cell, or, if from the same source, is modified from its original form. Thus, a heterologous gene in a prokaryotic host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a PTP) and a promoter. Additional factors necessary or helpful in effecting expression can also be used as described herein. For example, transcription termination signals, enhancers, and other nucleic acid sequences that influence gene expression, can also be included in an expression cassette.

As used herein, “contacting” has its normal meaning and refers to combining two or more agents (e.g., two proteins, a polynucleotide and a cell, etc.). Contacting can occur in vitro (e.g., two or more agents [e.g., a test compound and a cell lysate] are combined in a test tube or other container) or in situ (e.g., two polypeptides can be contacted in a cell by coexpression in the cell, of recombinant polynucleotides encoding the two polypeptides), in a cell lysate”

Various biochemical and molecular biology methods referred to herein are well known in the art, and are described in, for example, Sambrook et al., Molecular Cloning: A Laboratory Manual, Cold Spring Harbor Press, N.Y. Second (1989) and Third (2000) Editions, and Current Protocols in Molecular Biology, (Ausubel, F. M. et al., eds.) John Wiley & Sons, Inc., New York (1987-1999).

### SCREENING FOR MODULATORS OF NMDA-R SIGNALING

The present invention provides methods for identifying compounds therapeutic for treatment of psychotic disorders, by inhibiting NMDA-R signaling through the STEP phosphatase. The NMDA-R modulators are identified by detecting the ability of an agent to inhibit an activity of STEP, which is capable of directly or indirectly dephosphorylating an NMDA-R. The modulated activities of the PTP include, but are not limited to, its phosphatase activity, its binding to NMDA-R, and its activity on ERK and PTKs.

In some aspects of the invention, a STEP isoform is used in screening methods where the isoform comprises the phosphatase domain of STEP, e.g. STEP 61; STEP 46; etc. In other embodiments, the isoforms of STEP lacking the phosphatase domain, e.g. STEP 20, etc. are of interest, e.g. as negative controls or for comparison; and for determining agents that interact with the non-catalytic portions of the enzyme.

In one aspect, NMDA-R modulators of the present invention are identified by monitoring their ability to affect phosphatase activity. As will be detailed below, STEP, the NMDA-R/STEP-containing protein complex, or cell lines that express STEP or NMDA-R/STEP-containing protein complex, are used to screen for STEP agonists and antagonists that modulate direct or indirect NMDA-R tyrosine dephosphorylation, e.g. in the presence of a protein tyrosine kinase in a signaling pathway with STEP and NMDA-R. An agent that enhances the ability of STEP to directly or indirectly dephosphorylate NMDA-R will result in a net decrease in the amount of phosphotyrosine, whereas an agent that inhibits the ability of STEP to directly or indirectly dephosphorylate NMDA-R will result in a net increase in the amount of phosphotyrosine.

In some embodiments, the ability of an agent to enhance or inhibit STEP phosphatase activity is assayed in an in vitro system. In general, the in vitro assay format involves adding an agent to STEP (or a functional derivative of STEP) and a substrate of STEP, e.g. Src, Fyn, ERK, NMDA-R, etc., and measuring the tyrosine phosphorylation level of the substrate. In one embodiment, as a control, tyrosine phosphorylation level of the substrate is also measured under the same conditions except that the test agent is not present. By comparing the tyrosine phosphorylation levels of the substrate, PTP antagonists or agonists can be identified. Specifically, STEP antagonist is identified if the presence of the test agent results in an increased tyrosine phosphorylation level of the substrate. Conversely, a decreased tyrosine phosphorylation level in the substrate indicates that the test agent is a STEP agonist. The invention provides the use of such agents to modulate NMDA-R activity.

STEP used in the assays is obtained from various sources. In some embodiments, STEP used in the assays is purified from cellular or tissue sources, e.g., by immunoprecipitation with specific antibodies. In other embodiments, as described below, STEP is purified by affinity chromatography utilizing specific interactions of STEP with known protein substrates. In still other embodiments, STEP, either holoenzyme or enzymatically active parts of it, is produced recombinantly either in bacteria or in eukaryotic expression systems. The recombinantly produced variants of STEP can contain short protein tags, such as immunotags (HA-tag, c-myc tag, FLAG-tag), 6×His-tag, GST tag, etc., which could be used to facilitate the purification of recombinantly produced STEP using immunoaffinity or metal-chelation-chromatography, respectively.

Various substrates are used in the assays. Preferably, the substrate is Src, Fyn, ERK, NMDA-R, a functional derivative of NMDA-R, or the NR2A or NR2B subunit. In some embodiments, the substrates used are proteins purified from a tissue (such as immunoprecipitated NR2A or NR2B from rat brain). In other embodiments, the substrates are recombinantly expressed proteins. Examples of recombinant substrates include, but are not limited to, proteins expressed in E. coli, yeast, or mammalian expression systems. In still other embodiments, the substrates used are synthetic peptides that are tyrosine phosphorylated by specific kinase activity, e.g., Src or Fyn kinases.

Methods and conditions for expression of recombinant proteins are well known in the art. See, e.g., Sambrook, supra, and Ausubel, supra. Typically, polynucleotides encoding the phosphatase and/or substrate used in the invention are expressed using expression vectors. Expression vectors typically include transcriptional and/or translational control signals (e.g., the promoter, ribosome-binding site, and ATG initiation codon). In addition, the efficiency of expression can be enhanced by the inclusion of enhancers appropriate to the cell system in use. For example, the SV40 enhancer or CMV enhancer can be used to increase expression in mammalian host cells. Typically, DNA encoding a polypeptide of the invention is inserted into DNA constructs capable of introduction into and expression in an in vitro host cell, such as a bacterial (e.g., E. coli, Bacillus subtilus), yeast (e.g., Saccharomyces), insect (e.g., Spodoptera frugiperda), or mammalian cell culture systems. Mammalian cell systems are preferred for many applications. Examples of mammalian cell culture systems useful for expression and production of the polypeptides of the present invention include human embryonic kidney line (293; Graham et al., 1977, J. Gen. Virol. 36:59); CHO (ATCC CCL 61 and CRL 9618); human cervical carcinoma cells (HeLa, ATCC CCL 2); and others known in the art. The use of mammalian tissue cell culture to express polypeptides is discussed generally in Winnacker, FROM GENES TO CLONES (VCH Publishers, N.Y., N.Y., 1987) and Ausubel, supra. In some embodiments, promoters from mammalian genes or from mammalian viruses are used, e.g., for expression in mammalian cell lines. Suitable promoters can be constitutive, cell type-specific, stage-specific, and/or modulatable or regulatable (e.g., by hormones such as glucocorticoids). Useful promoters include, but are not limited to, the metallothionein promoter, the constitutive adenovirus major late promoter, the dexamethasone-inducible MMTV promoter, the SV40 promoter, and promoter-enhancer combinations known in the art.

The substrate may or may not be already in a tyrosine phosphorylated state (Lau & Huganir, J. Biol. Chem., 270: 20036-20041, 1995). In the case of a nonphosphorylated starting material, the substrate is typically phosphorylated, e.g., using an exogenous tyrosine kinase activity such as Src, or Fyn.

A variety of standard procedures well known to those of skill in the art are used to measure the tyrosine phosphorylation levels of the substrates. In some embodiments, a phosphotyrosine-recognizing antibody-based assay is used, e.g., radioimmunoassay (RIA), enzyme-linked immunosorbent assay (ELISA), as well as fluorescently labeled antibodies whose binding can be assessed from levels of emitted fluorescence. See, e.g., U.S. Pat. No. 5,883,110; Mendoza et al., Biotechniques. 27: 778-788, 1999. In other embodiments, instead of immunoassays, the substrates are directly labeled with a radioactive phosphate group using kinases that carry out selective tyrosine phosphorylation (Braunwaler et al., Anal. Biochem. 234:23-26, 1996). The rate of removal of radioactive label from the labeled substrate can be quantitated in liquid (e.g., by chromatographic separation) or in solid phase (in gel or in Western blots).

Comparing a tyrosine phosphorylation level under two different conditions (e.g., in the presence and absence of a test agent) sometimes includes the step of recording the level of phosphorylation in a first sample or condition and comparing the recorded level with that of (or recorded for) a second portion or condition.

In some embodiments of the invention, other than adding STEP to a substrate (e.g., NR2A or NR2B), the in vitro assays are performed with an NMDA-R/STEP-containing protein complex. Such protein complexes contain NMDA-R and STEP, or their functional derivatives. In addition, the complexes may also contain a PTK and other molecules. The NMDA-R/STEP-containing protein complexes may be obtained from neuronal cells using methods well known in the art, e.g., immunoprecipitation as described in Grant et al. (WO 97/46877). Tyrosine phosphorylation levels of the substrates are assayed with standard SDS-PAGE and immunoblot analysis.

In other embodiments, NMDA-R signaling modulators of the present invention are identified using in vivo assays. Such in vivo assay formats usually entail culturing cells co-expressing STEP and a substrate (e.g., NR2A or NR2B; e.g., recombinant forms of STEP and/or NMDA-R subunit substrate(s)), adding an agent to the cell culture, and measuring tyrosine phosphorylation level of the substrate in the cells. In one embodiment, as a control, tyrosine phosphorylation level of the substrate in cells not exposed to the test agent is also measured or determined. In some embodiments, the assay may be performed with non-neuronal cells expressing NR2A or NR2B, therefore in the absence of synaptic proteins.

In one embodiment, the in vivo screening system is modified from the method described in U.S. Pat. No. 5,958,719. Using this screening system, intact cells that express STEP and a substrate of STEP (e.g., Src, Fyn, ERK, NMDA-R, NR2A, or NR2B) are first treated (e.g., by NMDA) to stimulate the substrate phosphorylation. The cells are then incubated with a substance that can penetrate into the intact cells and selectively inhibit further phosphorylation (e.g., by a PTK) of the substrate, e.g. NMDA-R. The degree of phosphorylation of the substrate is then determined by, for example, disrupting the cells and measuring phosphotyrosine level of the substrate according to methods described above, e.g. with standard SDS-PAGE and immunoblot analysis. The activity of the PTP is determined from the measured degree of phosphorylation of the substrate. An additional measurement is carried out in the presence of an agent. By comparing the degrees of phosphorylation, agonists or antagonist of PTP that modulate NMDA-R tyrosine phosphorylation are identified.

In another embodiment, the present invention provides a method for identifying a nucleic acid molecule encoding a gene product that is capable of modulating the tyrosine phosphorylation level of NMDA-R. In one embodiment, a test nucleic acid is introduced into host cells coexpressing STEP and NMDA-R or their functional derivatives. Methods for introducing a recombinant or exogenous nucleic acid into a cell are well known and include, without limitation, transfection, electroporation, injection of naked nucleic acid, viral infection, liposome-mediated transport (see, e.g., Dzau et al., 1993, Trends in Biotechnology 11:205-210; Sambrook, supra, Ausubel, supra). The cells are cultured so that the gene product encoded by the nucleic acid molecule is expressed in the host cells and interacts with STEP and NMDA-R or their functional derivatives, followed by measuring the phosphotyrosine level of the NMDA-R. The effect of the nucleic acid on NMDA-R-signaling is determined by comparing NMDA-R phosphotyrosine levels measured in the absence or presence of the nucleic acid molecule.

It will be appreciated by one of skill in the art that modulation of binding of STEP and NMDA-R may also affect the level of tyrosine phosphorylation in NMDA-R by STEP. Therefore, agents identified from screening using the in vivo and in vitro assay systems described above may also encompass agents that modulate NMDA-R tyrosine phosphorylation by modulating the binding of STEP and NMDA-R. In some embodiments of the invention, NMDA-R modulators are identified by directly screening for agents that promote or suppress the binding of STEP and NMDA-R. Agents thus identified may be further examined for their ability to modulate NMDA-R tyrosine phosphorylation, using methods described above or standard assays well known in the art.

In one embodiment, modulators of the interaction between STEP and NR2A or NR2B are identified by detecting their abilities to either inhibit STEP and NMDA-R from binding (physically contacting) each other or disrupts a binding of STEP and NMDA-R that has already been formed. The inhibition or disruption can be either complete or partial. In another embodiment, the modulators are screened for their activities to either promote STEP and NMDA-R binding to each other, or enhance the stability of a binding interaction between STEP and NMDA-R that has already been formed. In either case, some of the in vitro and in vivo assay systems discussed above for identifying agents which modulate the NMDA-R tyrosine phosphorylation level may be directly applied or readily modified to monitor the effect of an agent on the binding of NMDA-R and STEP. For example, a cell transfected to coexpress STEP and NMDA-R or receptor subunit, in which the two proteins interact to form an NMDA-R/PTP-containing complex, is incubated with an agent suspected of being able to inhibit this interaction, and the effect on the interaction measured. Any of a number of means, such as coimmunoprecipitation, is used to measure the interaction and its disruption.

In one embodiment, the effect of modulators on the interaction between STEP and NMDA-R is observed through the detection of an increase in the surface expression of NMDA-R subunits, NR1, NR2A and NR2B. Such in vivo assay formats usually entail culturing cells co-expressing STEP and a substrate (e.g., NMDA-R), adding an agent to the cell culture, and measuring the surface expression of the NR1, NR2A and NR2B subunits. The surface expression can be determined using various methods well known in the art, e.g. immunoblots, immunocytochemistry, ELISA. In one embodiment, as a control, the surface expression of the NR1, NR2A and NR2B subunits in cells not exposed to the test agent is also measured or determined.

Although the foregoing assays or methods are described with reference to STEP isoforms and NMDA-R, the ordinarily skilled artisan will appreciate that functional derivatives or subunits of various STEP isoforms and NMDA-R may also be used. For example, in various embodiments, NR2A or NR2B is used to substitute for an intact NMDA-R in assays for screening agents that modulate binding of STEP and NMDA-R. In a related embodiment, an NMDA-R, ERK, Src, Fyn, functional derivative is used for screening agents that modulate phosphatase activity.

Further, in various embodiments, functional derivatives of STEP that have amino acid deletions and/or insertions and/or substitutions (e.g., conservative substitutions) while maintaining their catalytic activity and/or binding capacity are used for the screening of agents. A functional derivative is prepared from a naturally occurring or recombinantly expressed STEP isoform by proteolytic cleavage followed by conventional purification procedures known to those skilled in the art. Alternatively, the functional derivative is produced by recombinant DNA technology by expressing only fragments or combinations of exons of STEP in suitable cells. In one embodiment, a partial NMDA receptor or phosphatase polypeptide is expressed as a fusion polypeptide. It is well within the skill of the ordinary practitioner to prepare mutants of naturally occurring NMDA; or STEP isoforms that retain the desired properties, and to screen the mutants for binding and/or enzymatic activity. NR2A and NR2B derivatives that can be dephosphorylated typically comprise the cytoplasmic domain of the polypeptides, e.g., the C-terminal 900 amino acids or a fragment thereof.

In some embodiments, cells expressing STEP and NMDA-R may be used as a source of the STEP and/or NMDA-R, crude or purified, or in a membrane preparation, for testing in these assays. Alternatively, whole live or fixed cells may be used directly in those assays. Methods for preparing fixed cells or membrane preparations are well known in the art, see, e.g., U.S. Pat. No. 4,996,194. The cells may be genetically engineered to coexpress STEP and NMDA-R. The cells may also be used as host cells for the expression of other recombinant molecules with the purpose of bringing these molecules into contact with STEP and/or NMDA-R within the cell.

### THERAPEUTIC APPLICATIONS AND PHARMACEUTICAL COMPOSITIONS

NMDA-R antagonists can be used to treat psychotic symptoms caused by abnormal NMDA-R signaling. As discussed in detail below, the present invention provides pharmaceutical compositions containing STEP antagonists that modulate NMDA-R tyrosine phosphorylation. Such antagonists include, but are not limited to, agents that interfere with STEP gene expression, agents that modulate the ability of STEP to bind to NMDA-R or to dephosphorylate NMDA-R. In one embodiment, STEP antisense oligonucleotide or siRNA is used as STEP antagonist in the pharmaceutical compositions of the present invention. In addition, STEP inhibitors that inhibit dephosphorylation of NMDA-R are useful as NMDA-R signaling modulators.

NMDA-R hypofunction is causatively linked to schizophrenic symptoms (Tamminga, Crit. Rev. Neurobiol. 12: 21-36, 1998; Carlsson et al., Br. J. Psychiatry Suppl.: 2-6, 1999; Corbett et al., Psychopharmacology (Berl). 120: 67-74, 1995; Mohn et al., Cell 98: 427-436, 1999). In addition, NMDA-R hypofunction is also linked to psychosis and drug addiction (Javitt & Zukin, Am J Psychiatry. 148: 1301-8, 1991).

Using a STEP antagonist (NMDA-R agonists) as described herein, the present invention provides methods for the treatment of schizophrenia, and other psychoses by antagonizing the activity of STEP, by inhibiting the interaction between STEP and the NR2A or NR2B subunit; by interfering with the interaction between STEP and protein tyrosine kinases, by down-regulating expression of STEP, and the like.

It is well known in the art that NMDA-R agonists and antagonists can be used to treat neurologic disorders caused by abnormal NMDA-R signaling, e.g. acute insult of the central nervous system (CNS). Methods of treatment using pharmaceutical composition comprising NMDA agonists and/or NMDA antagonists have been described, e.g., in U.S. Pat. No. 5,902,815. As discussed in detail below, the present invention provides pharmaceutical compositions containing STEP antagonists and/or agonists that modulate NMDA-R tyrosine phosphorylation or downstream NMDA-R signaling. Such agonists and antagonists include, but are not limited to, agents that interfere with STEP gene expression, agents that modulate the ability of STEP to bind to NMDA-R or to dephosphorylate NMDA-R. In one embodiment, a STEP antisense oligonucleotide is used as a STEP antagonist in the pharmaceutical compositions of the present invention. In addition, STEP inhibitors that inhibit dephosphorylation of NMDA-R are useful as NMDA-R signaling modulators.

Abnormal NMDA-R activity elicited by endogenous glutamate is implicated in a number of important CNS disorders. In one aspect, the present invention provides modulators of STEP that, by modulating phosphotyrosine level of NMDA-R, can treat or alleviate symptoms mediated by abnormal NMDA-R signaling. Indications of interest include mild cognitive impairment (MCI), which can progress to Alzheimer's disease (AD). Treatment with acetyicholinesterase inhibitors can provide for modest memory improvement. Cognitive enhancers may also find use for memory loss associated with aging, and in the general public.

One important use for NMDA antagonist drugs involves the ability to prevent or reduce excitotoxic damage to neurons. In some embodiments, the STEP agonists of the present invention, which promote the dephosphorylation of NMDA-R, are used to alleviate the toxic effects of excessive NMDA-R signaling. In certain other embodiments, STEP antagonists of the present invention, which function as NMDA-R agonists, are used therapeutically to treat conditions caused by NMDA-R hypo-function, i.e., abnormally low levels of NMDA-R signaling in CNS neurons. NMDA-R hypofunction can occur as an endogenous disease process. It can also occur as a drug-induced phenomenon, following administration of an NMDA antagonist drug. In some related embodiments, the present invention provides pharmaceutical compositions containing STEP antagonists that are used in conjunction with NMDA antagonists, e.g., to prevent the toxic side effects of the NMDA antagonists.

Excessive glutamatergic signaling is causatively linked to the excitotoxic cell death during an acute insult to the central nervous system such as ischemic stroke (Choi et al., Annu Rev Neurosci. 13: 171-182, 1990; Muir & Lees, Stroke 26: 503-513, 1995). Excessive glutamatergic signaling via NMDA receptors has been implicated in the profound consequences and impaired recovery after the head trauma or brain injury (Tecoma et al., Neuron 2:1541-1545, 1989; McIntosh et al., J. Neurochem. 55:1170-1179, 1990). NMDA receptor-mediated glutamatergic hyperactivity has also been linked to the process of slow degeneration of neurons in Parkinson's disease (Loopuijt & Schmidt, Amino Acids, 14: 17-23, 1998) and Huntington's disease (Chen et al., J. Neurochem. 72:1890-1898, 1999). Further, elevated NMDA-R signaling in different forms of epilepsy have been reported (Reid & Stewart, Seizure 6: 351-359,1997).

Accordingly, STEP agonists of the present invention are used for the treatment of these diseases or disorders by stimulating the NMDA receptor-associated phosphatase activity or by promoting the binding of STEP to the NMDA receptor complex.

The STEP agonists (NMDA-R antagonists) of the present invention can also be used to treat diseases where a mechanism of slow excitotoxicity has been implicated (Bittigau & Ikonomidou, J. Child. Neurol. 12: 471-485, 1997). These diseases include, but are not limited to, spinocerebellar degeneration (e.g., spinocerebellar ataxia), motor neuron diseases (e.g., amyotrophic lateral sclerosis (ALS)), mitochondrial encephalomyopathies. The STEP agonists of the present invention can also be used to alleviate neuropathic pain, or to treat chronic pain without causing tolerance or addiction (see, e.g., Davar et al., Brain Res. 553: 327-330, 1991).

NMDA-R hypofunction have been causatively linked to various forms of cognitive deficiency, such as dementias (e.g., senile and HIV-dementia) and Alzheimer's disease (Lipton, Annu. Rev. Pharmacol. Toxicol. 38:159-177, 1998; Ingram et al., Ann. N.Y. Acad. Sci. 786: 348-361, 1996; Müller et al., Pharmacopsychiatry. 28:113-124, 1995). In addition, NMDA-R hypofunction is also linked to psychosis and drug addiction (Javitt & Zukin, Am J Psychiatry. 148: 1301-8, 1991). Further, NMDA-R hypofunction is also associated with ethanol sensitivity (Wirkner et al., Neurochem. Int. 35: 153-162, 1999; Yagi, Biochem. Pharmacol. 57: 845-850, 1999). NMDA-R hypofunction has also been linked to depression.

Using a STEP antagonist (NMDA-R agonists) described herein, the present invention provides methods for the treatment of Schizophrenia, psychosis, cognitive deficiencies, drug addiction, and ethanol sensitivity by antagonizing the activity of the NMDA-R-associated STEP, or by inhibiting the interaction between STEP and the NR2A or NR2B subunit.

The STEP antagonists of the present invention are directly administered under sterile conditions to the host to be treated. However, while it is possible for the active ingredient to be administered alone, it is often preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious to the patient. For example, the bioactive agent may be complexed with carrier proteins such as ovalbumin or serum albumin prior to their administration in order to enhance stability or pharmacological properties such as half-life. Furthermore, therapeutic formulations of this invention are combined with or used in association with other therapeutic agents.

The therapeutic formulations are delivered by any effective means that could be used for treatment. Depending on the specific STEP antagonist/NMDA-R agonist being used, the suitable means include but are not limited to oral, rectal, nasal, pulmonary administration, or parenteral (including subcutaneous, intramuscular, intravenous and intradermal) infusion into the bloodstream.

Therapeutic formulations are prepared by any methods well known in the art of pharmacy. See, e.g., Gilman et al (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa.; Avis et al (eds.) (1993) Pharmaceutical Dosage Forms: Parenteral Medications Dekker, N.Y.; Lieberman et al. (eds.) (1990) Pharmaceutical Dosage Forms: Tablets Dekker, N.Y.; and Lieberman et al (eds.) (1990) Pharmaceutical Dosage Forms: Disperse Systems Dekker, N.Y. The therapeutic formulations can conveniently be presented in unit dosage form and administered in a suitable therapeutic dose. The preferred dosage and mode of administration of a STEP antagonist will vary for different patients, depending upon factors that will need to be individually reviewed by the treating physician. As a general rule, the quantity of a STEP antagonist administered is the smallest dosage that effectively and reliably prevents or minimizes the conditions of the patients.

A suitable therapeutic dose is determined by any of the well known methods such as clinical studies on mammalian species to determine maximum tolerable dose and on normal human subjects to determine safe dosage. In human patients, since direct examination of brain tissue is not feasible, the appearance of hallucinations or other psychotomimetic symptoms, such as severe disorientation or incoherence, should be regarded as signals indicating that potentially neurotoxic damage is being generated in the CNS by an NMDA-R antagonist. Additionally, various types of imaging techniques (such as positron emission tomography and magnetic resonance spectroscopy, which use labeled substrates to identify areas of maximal activity in the brain) may also be useful for determining preferred dosages of NMDA-R agonists for use as described herein.

It is also desirable to test rodents or primates for cellular manifestations in the brain, such as vacuole formation, mitochondrial damage, heat shock protein expression, or other pathomorphological changes in neurons of the cingulate and retrosplenial cerebral cortices. These cellular changes can also be correlated with abnormal behavior in lab animals.

Except under certain circumstances when higher dosages may be required, the preferred dosage of STEP agonist and/or antagonist will usually lie within the range of from about 0.001 to about 1000 mg, more usually from about 0.01 to about 500 mg per day. It should be understood that the amount of any such agent actually administered will be determined by a physician, in the light of the relevant circumstances that apply to an individual patient (including the condition or conditions to be treated, the choice of composition to be administered, including the particular PTP agonist or the particular PTP antagonist, the age, weight, and response of the individual patient, the severity of the patient's symptoms, and the chosen route of administration). Therefore, the above dosage ranges are intended to provide general guidance and support for the teachings herein, but are not intended to limit the scope of the invention.

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which scope will be determined by the language in the claims.

It must be noted that as used herein and in the appended claims, the singular forms “a”, “and”, and “the” include plural referents unless the context clearly dictates otherwise. Thus, for example, reference to “a mouse” includes a plurality of such mice and reference to “the cytokine” includes reference to one or more cytokines and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for all relevant purposes, e.g., the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Characterization of STEP and NMDA-R Distribution

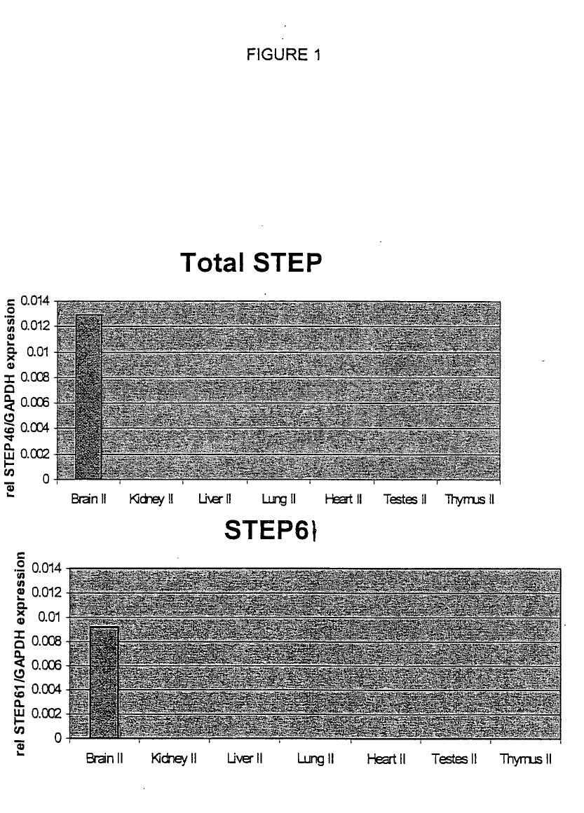

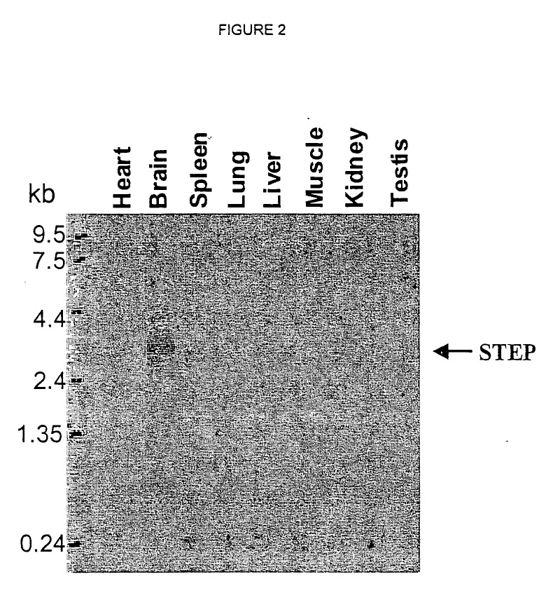

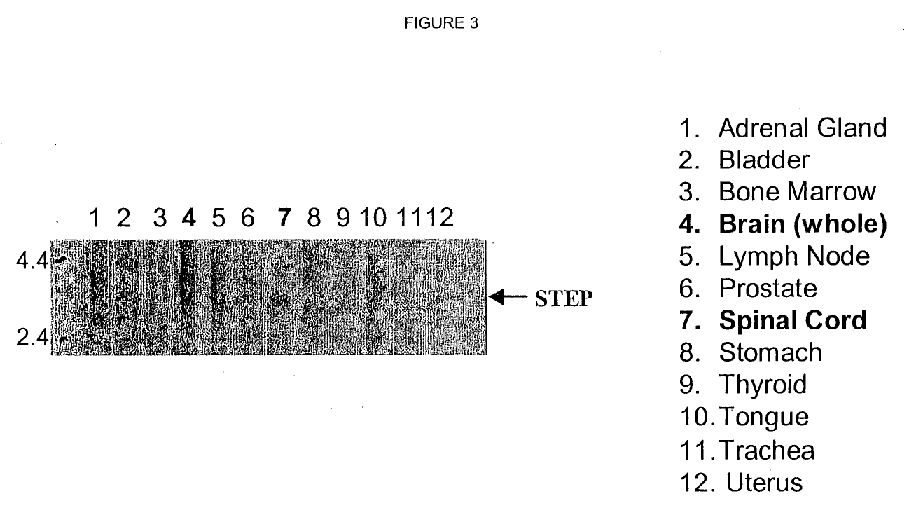

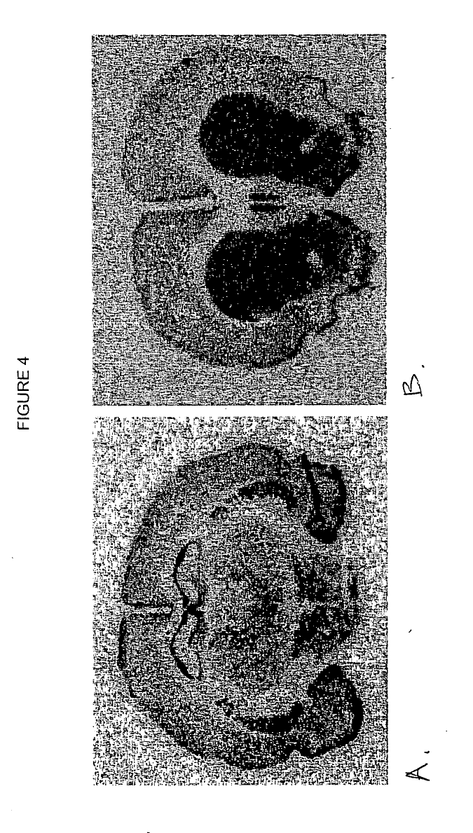

We have demonstrated that STEP is specifically expressed in the brain by quantitative PCR (FIG. 1), in rat tissues by Northern blot (FIG. 2) and in the human central nervous system (FIG. 3). Schizophrenia is associated with abnormalities in CNS function, and STEP is expressed in regions that are involved in schizophrenia. By in situ hybridization it is shown STEP is expressed in an interesting pattern in the brain (FIG. 4), that indicates a connection between STEP and schizophrenia. Schizophrenic brains show abnormalities in a number of brain regions including cortical areas, hippocampus, amygdala and striatum which are connected by glutamatergic circuits (references within Johnson et al, 1999) and thus from our data, STEP is expressed in areas abnormal in schizophrenia.

Quantitative PCR was performed by standard means. SYBR Green real-time PCR amplifications were performed in an icycler Real-Time Detection System (Bio-Rad Laboratories, Hercules, Calif.). The reactions were performed in duplicates in 25-μl reaction volume with the following PCR conditions: 50° C. for 2 minutes and 95° C. for 10 minutes, followed by 45 cycles of 95° C. for 15 seconds, 60° C. for 30 seconds followed by 72° C. for 40 seconds. Primers for Q-PCR were designed using Primer 3 software. Glyceraldehyde-3-phosphate dehydrogenase (GAPDH) was used an internal reference to normalize the target transcripts and relative differences were calculated using the PCR efficiencies according to Pfaffl (Pfaffl M. W. (2001) Nucleic Acids Res. 29(9):e45).

The expression pattern of STEP was also determined by Northern blotting. Multiple tissue Northern blots of rat and human origin were purchased from commercial sources. The membranes were prehybridized in 7% SDS, 0.5 M NaHP04, 1 mM EDTA at 65° C. for 15 minutes. Using fresh prehybridization solution, the membranes were hybridized with the labeled probe for 18 hours. The hybridized membranes were briefly rinsed in 5% SDS, 40 mM NaHPO4, 1 mM EDTA and then washed for 45 minutes at 65° C. with fresh solution. This wash solution was replaced with 1% SDS, 40 mM NaHPO₄, 1 mM EDTA and washed twice for 45 minutes at 65° C. with fresh solution. After washing, the membranes were sandwiched between plastic wrap and exposed overnight to Kodak X-OMAT AR film with a Dupont Lightening Plus intensifying screen at −70° C.

The results are shown in FIG. 2 for rat tissues and FIG. 3 for human tissues. The size of the predominant STEP mRNA is 3 kb.

In situ hybridization was performed by standard methods. Animals included in the in situ hybridization experiment were terminated by decapitation. The brains were removed and placed in a plastic form with the embedding material and frozen on a mixture of dry ice and ethanol. The frozen blocks were stored at −80 C before sectioning.

Rat brain coronal sections were cut at 14.5 μm thick sections on a Microm cryostat at −17° C. and thaw-mounted on positively charged slides and dried at room temperature for 10 minutes before storage in −80° C. freezer. The pre-hybridization of slides were started by fixation in 4% ice-cold paraformaldehyde for 10 minutes followed by 5 minutes rinse in 1× ice-cold 0.1 mol/L phosphate buffer saline (PBS pH 7.2). The sections were then processed as followed: washed for 1 minute in 0.1 mol/L TEA and for 10 minutes in 0.25% acetic anhydride\TEA. Rinsed 2 times in 1×SSC and dehydrated in 70% (two minutes), 95% (two minutes) and 100% (two minutes) ethanol. Finally the sections were incubated for 5 minutes in 100% chloroform followed by 2 minutes incubation in 95% ethanol. The slides were finally air-dried for 10 minutes before hybridization.

Probe Generation and hybridization: A linear DNA with transcription sites SP6 and T7 was generated using PCR amplification. 1 μg of the PCR fragment with SP6 and T7 were used as template for in vitro transcription. UTP [α-³³P] (NEN) were used to generate a hot sense and anti sense riboprobe by in vitro transcription using T7 and SP6 polymerases. The sections were then. probed with 200 μL hybridization cocktail with 10⁵ cpm specific activity, covered with coverslips and placed in a humidified chamber for 18 hours at 55° C. Hybridization cocktail in addition to pre-labeled probe consisted of 50% formamide, 0.3 mol/L standard saline, 1× Denhardt's solution, 0.01 mol/L DTT, 0.01 mol/L Tris, 10% Dextran sulfate and 0.001 mol/L EDTA. For each hot probe a cold probe was also generated to test the specificity of the riboprobe, competition experiments were carried out by adding unlabelled probe at 100 times the concentration of the labeled one which abolished the binding of the STEP probe. Also, when the hot sense probe of STEP was tested no specific binding to the tissue was detected. After overnight hybridization the sections were rinsed in 1×SSC at room temperature. The sections were then treated for 30 minutes with RNAase A (10 g/L) in RNAase buffer consisting of 0.01 mol/L Tris (pH 8.0), 0.5 mol/L NaCl and 0.001 mol/L EDTA at 37° C. The RNAase A treatment was followed by 30 minutes rinse in RNAase buffer at 37° C., 15 minutes at 1×SSC at room temperature and finally 0.5×SSC at 65° C. for 30 minutes. After last wash in 0.5×SSC, the slides were dehydrated in 70% (2 minutes), 95% (2 minutes) and 100% (2 minutes) ethanol and finally air-dried for 10 minutes and were exposed to the phosphoimager screens (Cyclone) for 5-7 days at room temperature. After 7 days of exposure the phosphoimager screens were scanned. Images obtained are presented in FIG. 4, STEP is expressed highly in the striatum and hippocampus and at appreciable levels in other brain regions including the cortex and thalamus.

### Example 2

### Characterization of STEP Effects on NMDA-R Function in a Heterologous Expression System

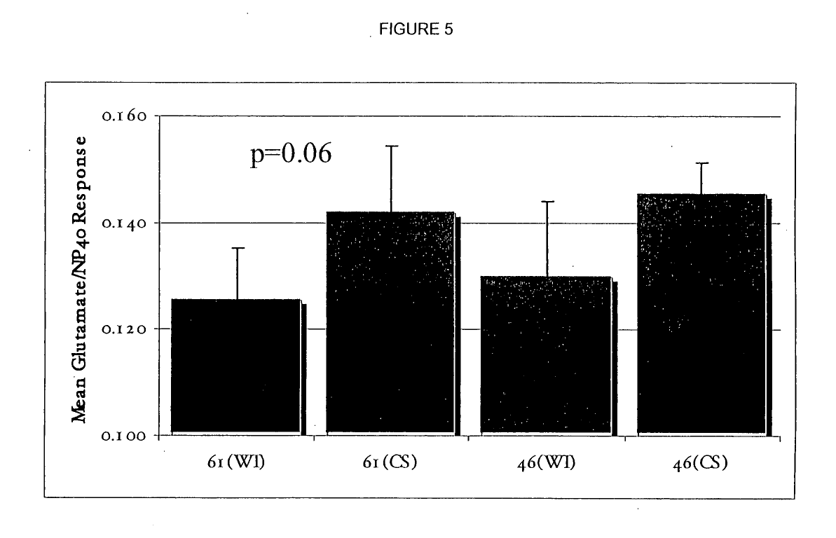

NMDA receptor hypoactivity has been linked to schizophrenia (Coyle et. al., 2002) and NMDA receptor antagonists can exacerbate schizophrenic symptoms (Lahti et al, 1995). We have found that STEP reduces NMDAR function by its effects on the Ca influx through NMDARs in transfected HEK293 cells that stably express NMDARs (FIG. 5).

Cell lines were used that stably express the NR1 subunit under the control of a tetracycline inducible element and the NR2B subunit constitutively. These cell lines were transiently transfected with one of the following constructs using Fugene:
STEP61
STEP61(CS)—a catalytically inactive form of STEP61 in which the residue critical for phosphatase activity, cysteine-300, was mutated to a serine.
STEP46
STEP46(CS)—a catalytically inactive form of STEP46 in which the residue critical for phosphatase activity, cysteine-172, was mutated to a serine.

One day after transfection cells were transferred to a 96 well, black walled, clear bottom, assay plate and expression of NMDA receptors was induced by addition of tetracycline. One day later the function of NMDA receptors in the presence of the STEP constructs was assessed. Cells were washed with assay buffer (Hepes buffered saline solution supplemented with 5 mM HEPES, 10 μM glycine and 1 mM calcium chloride) and loaded with a derivative of fluo-3 in assay buffer for 1 hour at 37° C. The assay plate was transferred to a Molecular Devices FLEXstation, a scanning fluorometer coupled with a fluid transfer system that allows the measurement of rapid, real time fluorescence changes in response to application of compounds. Baseline measurements of fluorescence were obtained by taking baseline readings every 1.5 seconds for 30 seconds. Glutamate at a final concentration of 1 μM was added and fluorescence readings taken every 1.5 seconds for a further 2 minutes. At this time NP40 at a final concentration of 1% was added and readings were taken for a further 30 seconds. The peak response to glutamate was measured and divided by the peak response to NP40 to assess normalized glutamate induced calcium influx into the cells for each construct. Comparison of the different constructs indicated that inactive mutants show lower NMDA receptor function by calcium flux measurement than active forms of STEP (FIG. 5).

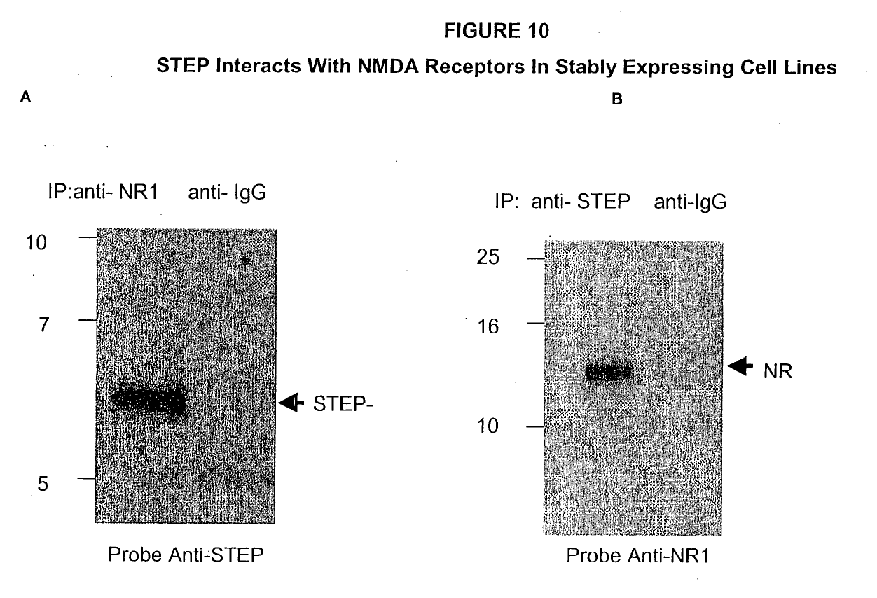

STEP, and STEP-61 interact with NMDA receptors even in the absence of other synaptic proteins, as shown in FIGS. 10. Cell lines were used that stably express the NR1 subunit under the control of a tetracycline inducible ejement and the NR2B subunit constitutively. These cell lines were further stably infected with STEP using lentivirus mediated gene delivery.

Stably transfected cell lines that express NR1, NR2B and STEP constructs were isolated and confirmed by immunostaining and Western blotting. NR1/NR2B/STEP cell lines were plated on cell culture dishes and expression of NMDA receptors was induced by addition of tetracycline. One day later the cells were harvested for immunoprecipitation experiments.

Immunopreciptation: Cells were harvested, the medium removed upon centrifugation and the cells resuspended in Lysis Buffer (150 mM NaCl, 50 mM Tris pH 7.6, 1% Triton). 2000 μg lysate (1 μg/μl) is incubated with 5-10 μg of primary antibody, overnight at 4° C., shaking.

After incubation of antibodies, 100 μl of Protein A/G-Agarose (Amersham) slurry is added, and the incubation is continued for another hour. To determine immunoprecipitated proteins, material bound to Protein AG Agarose is separated by pelleting the beads with the immunocomplex attached by centrifugation, washed with PBS and resolved by SDS-PAGE. Proteins resolved on the gel are transferred to membrane to verify the presence of co-immunoprecipitated proteins by Western blots using specific antibodies. Anti-NR1 antibody and a monoclonal Anti-STEP antibody (Novus Biologicals Clone #23E5, Cat # NB300-202) was used as probes (FIG. 10).

The data shows that NR1 co-precipitates with STEP. Co-immunoprecipitation experiments were performed to further identify the subunit specificity of the physical interaction between NMDA-R and STEP.

HEK-293 cells were transfected with constructs for expression of either STEP-46, STEP-61, NR1, NR2A or NR2B or a combination of these using Fugene. Two days after transfection cells were harvested and used for immunoprecipitation. Cells were harvested, the medium removed upon centrifugation and the cells resuspended in Lysis Buffer (150 mM NaCl, 50 mM Tris pH 7.6, 1% Triton). 2000 μg lysate (1 μg/μl) is incubated with 1-3 μg of primary antibody, overnight at 4° C., shaking. Immunoprecipitation was performed using an appropriate antibody to each NMDA subunit transfected and the interaction with NMDA subunits

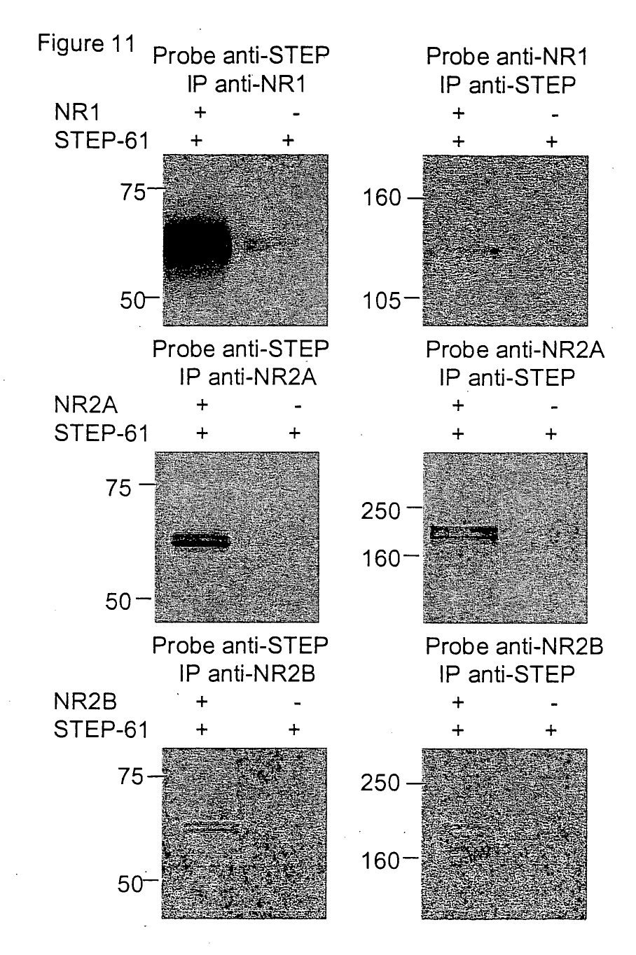

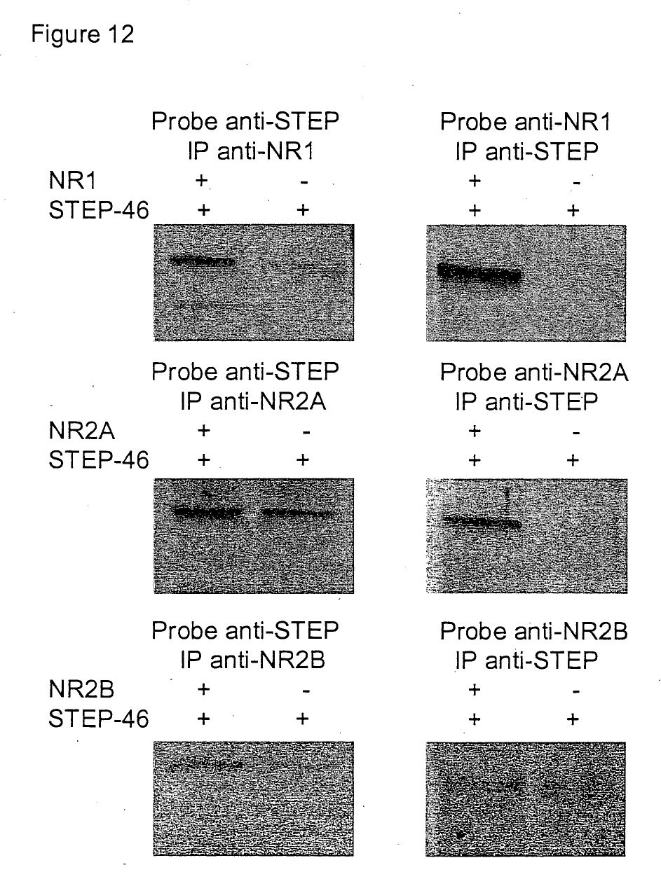

After incubation of antibodies, 100 μl of Protein A/G Agarose (Amersham) slurry is added, and the incubation is continued for another hour. To determine immunoprecipitated proteins, material bound to Protein AG Agarose is separated by pelleting the beads with the immunocomplex attached by centrifugation, washed with PBS and resolved SDS-PAGE. Proteins resolved on the gel are transferred to membrane to verify the presence of co-immunoprecipitated proteins by Western blots using anti-STEP antibody. The data shows that NR1, NR2A and NR2B co-precipitate with STEP (FIG. 11 and FIG. 12).

Both STEP61 (FIG. 11) and STEP 46 (FIG. 12) are able to interact with NMDAR. Therefore both major forms of STEP expressed in the brain are able to interact with and modulate the function of NMDAR. Furthermore both STEP46 and STEP61 are able to interact with NR1, NR2A and NR2B subunits. Therefore STEP is able to interact with all forms of NMDAR present in the adult brain. The significance of this is that STEP acts universally in all brain regions and on all NMDA receptors in the brain and can influence function of all NMDA receptors.

### Example 3

### Characterization of STEP Effects on NMDA-R Function in Cultured Cortical Neurons

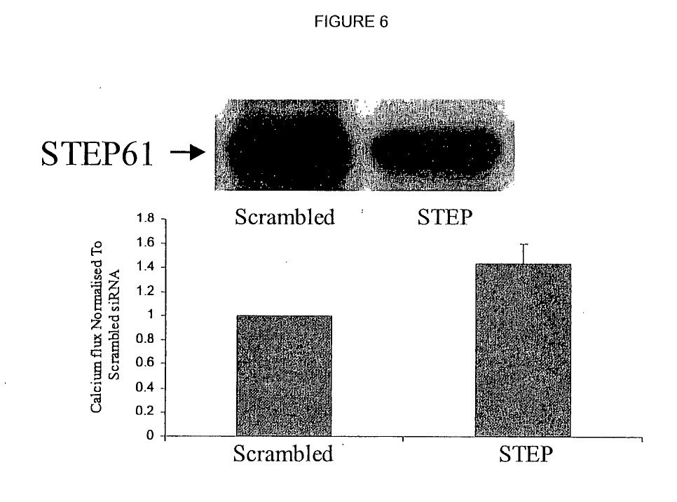

The use of RNAi to reduce STEP levels in cultured cortical neurons causes an increase in NMDA receptor mediated Ca influx into neurons (FIG. 6). This suggests that STEP actively causes a decrease in NMDAR function in neurons, which could lead to NMDAR hypoactivity and hence schizophrenia.

Single stranded interfering RNA molecules (RNAi) were designed to be complementary to the sequence of STEP by standard means. The sequence used was 5′ AAA CAU GCG AAC AGU AUC AGU 3′. A standard scrambled RNA molecule of sequence 5′-CAG TCG CGT TTG CGA CTG G-3′ was used as a control. Dissociated cortical neurons were prepared from E18 rat embryos by standard protocols. The dissociated neurons were mixed with 90 ul of rat Nucleofector solution to give a final concentration of 4.8×10⁶ cells/90 ul. The cells were mixed with 20 ug of RNAi and transferred to an electroporation cuvette. Using an AMAXA Nucleofector cells were electroporated using standard settings. Cells were transferred from the electroporation cuvette to poly-D-lysine coated 96 well plates for calcium flux assays or 6 well plates for Western blotting procedures and grown in standard neuronal media at 37° C. with 5% CO₂ for 4 days.

Knockdown of endogenous STEP protein levels was visualized by Western blotting. Four days after electroporation cells were harvested and lysed with lysis buffer (150 mM NaCl, 50 mM Tris pH 7.6, 1% triton, in the presence of a protease inhibitor cocktail and 1 mM sodium orthovanadate) on ice. Protein samples were separated by SDS-polyacrylamide gel electrophoresis and proteins transferred to nitrocellulose membranes. Levels of STEP protein were determined by Western blotting using anti-STEP antibodies (FIG. 6).

Functional experiments were performed using the Molecular Devices FLEXstation as described previously. To work with neuronal cultures buffers were supplemented with 1 μM tetrodotoxin and 100 nM nifedipine and specific activation of NMDA receptors was achieved by applying 1 μM NMDA instead of glutamate.

Analysis of NMDA mediated calcium influx indicates that when STEP protein levels are reduced by RNA interference there is an increased NMDA mediated calcium influx into cultured cortical neurons (FIG. 6).

### Example 4

### Surface Eexpression of NMDA Receptors is Specifically Increased by Knockdown of STEP Levels by RNA Interference

Interfering RNA molecules were introduced into neurons by AMAXA nucleofection. To specifically knockdown STEP the oligo used had the sequence 5′-AAACAUGCGMCAGUAUCAGU-3′, a control siRNA that is predicted not to knock down levels of any gene products (sequence 5′-UAGCGACUAAACACAUCAAUU-3′ was also used (Dharmacon, Lafayette, Colo.). Interfering RNA molecules were introduced into neurons using the AMAXA nucleofection technique (AMAXA, Gaithersburg, Md.) using the manufacturers protocol. Neurons were allowed to grow in culture for 3-4 days prior to experimental procedures.

Neurons were washed with ice cold PBS. Sulfo-NHS-LC-Biotin (Pierce Biotechnology, Rockford, Ill.) was dissolved at 1.5 mg/ml in ice cold PBS and incubated with the cells for 1 hour at 40° C. Excess biotin was washed off the cells with ice cold PBS and cells lysed and harvested as above. 100 mg of protein lysate was incubated with 100 μl of neutravadin agarose slurry (Pierce Biotechnology) overnight at 40° C. The complexes were washed thoroughly and proteins eluted from the beads by resuspension in SDS loading buffer and boiling for 10 mins. Proteins were loaded on 8% Tris-gicine gels and separated by SDS-PAGE. Western blotting was performed by standard means using the indicated antibodies.

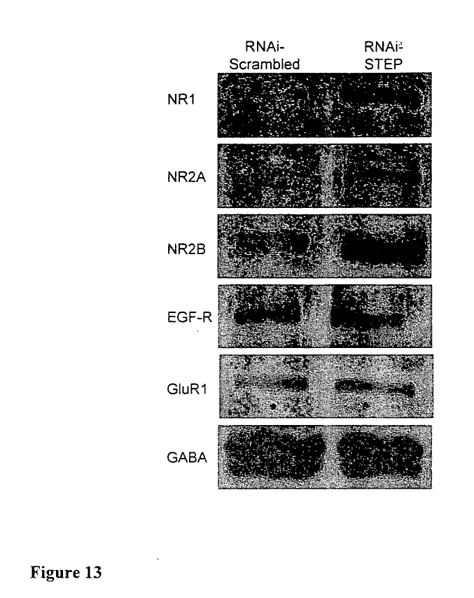

Results show there is a specific increase in NMDA receptor surface expression (as assessed by NR1, NR2A and NR2B levels at the surface membrane) upon knockdown of STEP levels by RNA interference. Levels of the EGF receptor, GluR1 subunit of AMPA receptors or GABA_{A} Receptors was unaffected by modulation of STEP levels (FIG. 13).

### Example 5

### Characterization of STEP Effects on ERK Phosphorylation

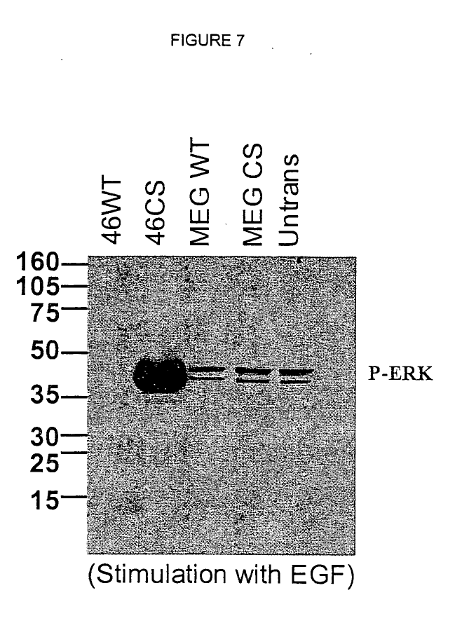

Stably expressing NMDA receptor HEK293 cell lines (as described previously) were transfected with STEP61, STEP61CS, STEP46 or STEP46CS constructs by standard means and grown in 6 well plates for two days. Cells were washed with PBS and then treated in the absence or presence of 50 ng/ml EGF (in PBS) for 15 minutes. Cells were then harvested on ice in lysis buffer (150 mM NaCl, 50 mM Tris pH 7.6, 1% Triton, in the presence of a protease inhibitor cocktail and 1 mM sodium orthovanadate) and lysed for 1 hour with shaking at 4° C. Solubilized proteins were separated by centrifugation and resolved by SDS-polyacrylamide gel electrophoresis. Proteins were transferred to nitrocellulose membranes and Western blotting was performed using antibodies that specifically recognize the phosphorylated form or ERK (Biosource). To ensure that samples were loaded equally antibodies were stripped form the membranes using stripping buffer (100M β-mercaptoethanol, 2% SDS, 62.5 mM Tris-HCl, pH6.7) at 55° C. for 30 minutes and membranes were reprobed with an antibody that recognizes total ERK levels. This demonstrated that there is much less phosphorylation of ERK in the presence of active forms of STEP compared to forms that have a mutation to disrupt their catalytic activity (FIG. 7).

ERK phosphorylation in cultured cortical neurons (10-13 division) can be elicited by activation of NMDARs. Neurons show low levels of basal ERK phosphorylation. Upon stimulation for 5 minutes with NMDA (100 μM) then is significant ERK phosphorylation observed. This effect is blocked by incubation with the competitive NMDA receptor antagonist D-2-amino-5-phosphonopentanoic acid (D-APV). Therefore in neurons a major pathway leading to ERK phosphorylation is via activation of NMDARs.

Cultured cortical neurons at 10 to 13 days in vitro were infected with Sindbis virus containing RNA encoding STEP61, STEP61CS (a catalytically inactive form of STEP61 in which the residue critical for phosphatase activity, cysteine-300, was mutated to a serine) or GFP (control). 150 ul of Sindbis virus that expresses GFP, STEP-61 or STEP61 (CS) were used to infect a 10 cm petri dish of neurons. Sindbis virus infection was allowed to proceed for 18 hours before stimulation and harvesting.

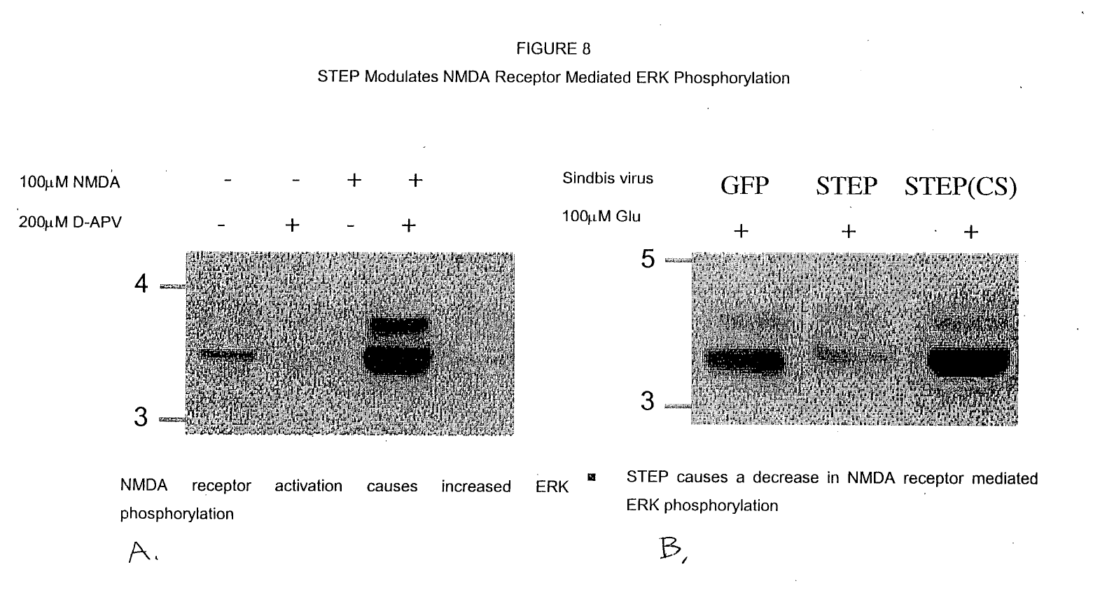

18 hours after infection neurons were washed with PBS and then treated with 100 μM glutamate for 5 minutes. Cells were then harvested, lysed and proteins separated and phospho-ERK levels detected by western blot. This demonstrated that there is much less phosphorylation of ERK in the presence of active forms of STEP compared to forms which have a mutation to disrupt their catalytic activity (FIG. 8).

### Example 6

### Characterization of STEP Effects on Protein Tyrosine Kinase Phosphorylation

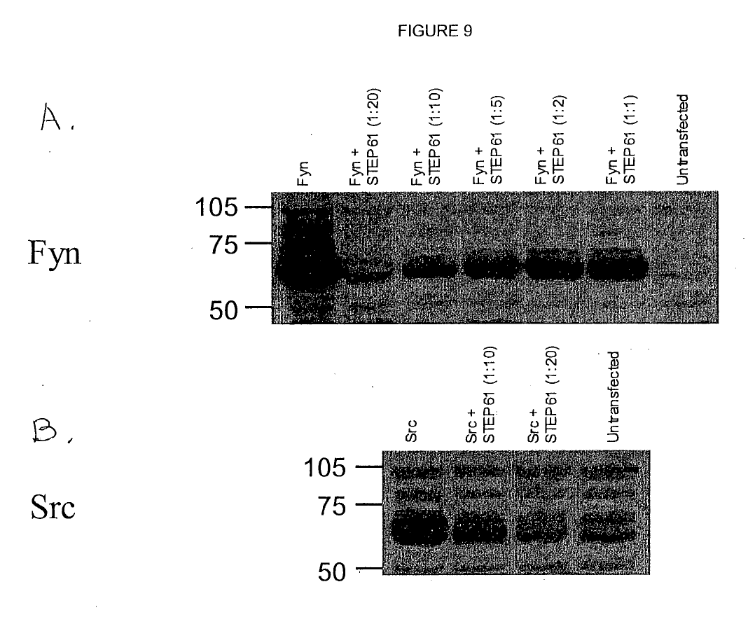

Stably expressing NMDA receptor HEK293 cell lines (as described previously) were transfected with a mutated form of src [Src(KP)] or Fyn [Fyn(Y531F)] in the presence or absence of varying concentrations of STEP61 by standard means and grown in 6 well plates for two days. Cells were then harvested on ice in lysis buffer (150 mM NaCl, 50 mM Tris pH 7.6, 1% triton, in the presence of a protease inhibitor cocktail and 1 mM sodium orthovanadate) and lysed for 1 hour with shaking at 4° C. Solubilised proteins were separated by centrifugation and resolved by SDS-polyacrylamide gel electrophoresis. Proteins were transferred to nitrocellulose membranes and Western blotting was performed using antibodies that specifically recognize the phosphorylated form or Src at the tyrosine 418 residue (which also recognizes Fyn phosphorylated at residue Y420). To ensure that samples were loaded equally antibodies were stripped from the membranes using stripping buffer (100M β-mercaptoethanol, 2% SDS, 62.5 mM Tris-HCl, pH6.7) at 55° C. for 30 minutes and membranes were reprobed with an antibody that recognizes total src or fyn levels. This demonstrated that there is a concentration dependent dephosphorylation of src and fyn at this critical site in the presence of STEP61 (FIG. 9).

### Example 7

### Screening for Agents that Modulate NMDA-R Signaling

STEP expression and purification. A 1.1 Kb DNA fragment encoding hSTEP46 (residues E2 through E369) preceded by the tag HHHHHH was subcloned into the pET-17b vector (Novagen) between the NdeI and HindIII sites. The resulting plasmid was transformed into both BL21(DE3) cells (Invitrogen) and Tuner(DE3) cells (Novagen), which were both used for large scale expressions of hSTEP46. Cells were grown in LB medium at 37° C. and induced at A₆₀₀ =0.6-1.0 with 0.1 mM IPTG for 6 hours before harvest.

The cell paste was sonicated in lysis buffer composed of 50 mM HEPES, pH 8.0, 0.3 M NaCl, 1 mM PMSF, 1 mM β-mercaptoethanol, and 0.1% Triton X-100. The cell lysate was centrifuged at 27,000×g for 20 min, and the supernatant was loaded onto a Ni²⁺-NTA (Qiagen) column equilibrated with 10 mM imidazole, 0.3 M NaCl, 50 mM HEPES, pH 8.0 buffer. The column was washed with the same buffer, and the protein was eluted with 250 mM imidazole, 0.3 M NaCl, 50 mM HEPES, pH 8.0 buffer.

The eluate from the Ni²⁺-NTA column was adjusted to 1 M ammonium sulfate and chromatographed on a Macro-Prep Methyl HIC (BioRad) column. The protein was eluted with 0.5 M ammonium sulfate and buffer exchanged into 50 mM HEPES, pH 7.5 buffer.

The protein obtained over the two chromatographies was at least 95% pure by Coomassie staining.

Assay Development

A number of in vitro assays are utilized to assess the activity of STEP and subsequently to screen for compounds that modulate its function. An example is TR-FRET, but to those skilled in the art alternative phosphatase activity assays will be evident.

TR-FRET Assay

Material:

Phosphatase Buffer 50 mm HEPES, pH 8; 1 mM DDT; 2 mM EDTA; 0.01% Brij solution; 10 mM MgCl₂. Detection Buffer: 25 mM Tris, pH 7.5+0.2% Trition 100; 0.5 μl Eu PY20 Ab; 1.5 μl Streptavidin-APC per 5 ml of Detection BufferSubstrate: AGY 1336. Enzyme: STEP. Sodium Orthovanadate. DMSO (HPLC grade). Compound Plates: Compound plates are thawed overnight at room temp.

Method:

The enzyme stock solution is made by adding 24.4 μl STEP stock to 100 ml of phosphatase buffer. The substrate stock solution is made by adding 2 μL AGY-1336 (at 5 mM) to 100 ml of phosphatase buffer. The control inhibitor stock solution is made by adding 90 μl sodium orthovanadate (100 μM) to 30 ml phosphatase buffer. The detection reagent stock solution is made by adding 15 μL Eu-anti-phosphotryosine antibody+45 μL APC to 150 ml of detection buffer. This yields initial concentrations of: Enzyme: 10 μM; substrate: 100 nM; vanadate: 300 nM.

The reagents for the control wells are dispensed by the Biomek 2000 (B2K) and Biomek FX robots. The B2K dispenses controls into six assay plates. 12.5 μl of enzyme, 2.5 μl of DMSO, and 10 μl of buffer is placed into column 1 and 2, rows A through H. A substrate volume of 12.5 μl, 2.5 μl of DMSO, and 10 μl of buffer is placed into columns 1 and 2, rows l through P. Column 23, row A through P will contain 5.0 μl of orthovanadate solution. Column 24 is left empty.

For the enzyme activity assay, 2.5 μl of compound, 12.5 μl of enzyme, and 10 μl of substrate (separated by air gaps) are added to columns 3 thru 24 by the Biomek FX in a single dispense. After the dispense, the tips are washed with DMSO and water for re-use between each quadrant. Once the assay plates are set up, they are incubated at 27° C. for 45 minutes. Then 20 μl of detection buffer is added to stop the reaction and to allow the Europium antibody (Eu-Ab) and streptavidin-APC to bind to the substrate.

The plates are then placed in the plate reader, an Analyst HT. Excitation light at 360 nm is used to excite the Europium antibody with an emission at 620 nm. Fluorescence resonance energy transfer (FRET) from Eu-Ab to APC will only occur when they are in close proximity. Therefore, when an APC emission is observed at 665 nm the enzyme has been inhibited from removing the phosphate group from the substrate. The FRET assay is time-resolved (TR), where there is a delay between excitation light and collection of emission signals. This reduces the amount of stray light created by short-lived fluorescing molecules. The Analyst HT measures APC and Europium emission signals and calculates the ratio between the two intensities. Typical intensities for the Europium is ˜2000 and APC is ˜600.

The specificity of inhibition is tested using a broad phosphatase panel to determine inhibition of phosphatases other than STEP. Once hits are identified as specific to STEP, the inhibitor is tested is secondary assays as described below, e.g. HEK293 cells expressing NR1/NR2A and NR1/NR2B subunits. Functional characterization of active compounds is performed in primary hippocampal neurons by electrophysiology. In vivo validation of STEP inhibitors uses behavioural tests in mouse or rat animal models.

Design of profiling assays. The development of secondary cell-based assays is used in the profiling of compounds. Key parameters of increased NMDAR activity including increased NR2 phosphorylation; increased NMDAR current; increased Ca²⁺ permeability, increased NMDAR surface expression are assessed. Transient expression of glutamate receptor subunits in HEK293 cells is used. The phosphorylation state of the NR2 subunits by endogenous kinases in HEK293 cells is determined, and tested for an effect on NMDA receptor activity.

The profiling assays include transient expression of binary NR1/NR2B and NR1/NR2A receptor channels in the presence and absence of the agonist glutamate. Stable cell lines may also be used. Glutamate, by activating the NMDA receptor channels, also leads to an increased phosphorylation (only in presence of activated PTK) of the NR2 subunits and thus to increased current and Ca²⁺ permeability. Identified compounds will specifically inhibit STEP and lead to increased NR2 phosphorylation and Ca²⁺ influx upon NMDAR activation with glutamate. The functionality of NMDA receptors and their modulation is initially tested using calcium flux measurements. Different calcium indicator dyes are assessed.

For profiling assays, primary hippocampal or cortical neurons are used uninfected or infected with either Sindbis or Lentivirus constructs expressing STEP, STEPCS and a GFP control. Organotypic cultures are also used. NMDA or L-Glutamate induced currents are recorded selectively in presence/absence of identified compounds. In order to measure NMDA currents, the cells are clamped with the patch pipette and characteristic NMDA-R currents recorded at different membrane potentials (Kohr & Seeburg, J. Physiol (London) 492:445-452, 1996).

Neuronal NMDA receptor function is measured using either electrophysiology or the FLEX station, i.e measuring Ca2+ influx. A calcium imaging experiment is carried out as follows. Measurements are done in presence/absence of compounds in a primary neuronal cell expressing NMDA-R subunits as described above by using a FLEX station/FLIPR or Ca²⁺ Imaging (see Renard, S. et al. Eur. J. Physicology 366:319-328 (1999)). The FLEX station in combination with calcium indicator dyes is used to measure NMDA receptor activity. Similarly to the experiments in HEK293, it is expected to see a decrease in NMDAR current in neurons infected with the wt STEP virus. Compounds would restore NMDAR function/activity by inhibiting STEP. The STEP (cs) mutant serves as a control.

Additional assays utilize the additional role of STEP in dephosphorylation of ERK and protein tyrosine kinases as described previously. Assays are performed using Western blotting or ELISA techniques to assess the effects of compounds on the phosphorylation state of these proteins which are substrates of STEPs either in heterologous expression systems or neuronal preparations.

Changes in the surface expression of NMDARs are also used as an assay to profile compounds. Cortical neurons are dissociated from E17-E18 rat embryos and grown as dissociated cultures for 4-5 days in vitro. STEP modulators are incubated with the neurons and surface levels of NMDARs assessed. Briefly, treated neurons are taken and the surface expressed receptors labeled with a non-cell permeable form of biotin (Sulfo-NHS-LC-Biotin), on ice for 30 mins. Excess biotin is washed off with ice cold PBS and the cells subsequently harvested and lysed to extract the proteins. Biotinylated proteins are precipitated by overnight incubation with neutravadin coupled to agarose. The formed complexes are washed thoroughly with cold PBS and the pulled-down, biotinylated proteins are eluted from the column in SDS containing buffer with boiling. The proteins are separated by SDS-polyacrylamide gel electrophoresis (SDS-PAGE) and transferred to nitrocellulose membranes by standard techniques. Membranes are probed with appropriate primary antibodies, for example against the NR1, NR2A or NR2B subunits, and subsequently with an appropriate HRP-conjugated secondary antibody. Standard chemiluminescent techniques are used to visualize the labeled proteins on membranes. Levels of surface expressed NMDARs can be quantitated and the effects of STEP modulators assessed.

### Example 8

### Prepulse Inhibition

Schizophrenia is a chronic and debilitating syndrome, which is generally associated with a wide range of cognitive and emotional alterations. One preclinical model for the disease is the prepulse inhibition paradigm. Prepulse inhibition (PPI) refers to the inhibition of a startle reflex that occurs when an intense startling stimulus (acoustic or tactile) is preceded by a barely detectable prepulse. PPI provides an operational measure of sensorimotor gating and may reflect the ability to screen exteroceptive stimuli for their physiological or cognitive relevance. Several clinical studies have shown that schizophrenic patients have deficient PPI and startle habituation (SH). Habituation is viewed as the simplest form of non-associative learning and reflects decreased responding to repeated presentation of an initially novel exteroceptive stimulus. Common neuropathological mechanisms have been proposed to underlie clinical signs and reduced PPI and habituation in the schizophrenic patients.

As shown by a number of studies, reliable startle reflex and PPI can be obtained in mice using stimulus parameters almost identical to those used in rats. More importantly, marked genetic differences in PPI are also reported across strains of mice, with the C57BL/6J strain showing a poor PPI. Thus, in the present study it is tested whether various doses of antipsychotics could improve PPI in mice showing poor sensorimotor gating.

Methods

Animals. Adult male mice of the following strains: C57BL/6Jare used. Animals weighing between 20 and 24 g are housed four per cage with water and food ad lib. They are allowed 1 week of acclimation prior to testing.

Apparatus. Testing is conducted in startle devices (SRLAB, San Diego Instruments, San Diego, Calif., USA) each consisting of a 5.1 cm (outside diameter) Plexiglas cylinder mounted on a Plexiglas platform in a ventilated, sound-attenuated cubicle with a high frequency loudspeaker (28 cm above the cylinder) producing all acoustic stimuli. The background noise of each chamber is 70 dB. Movements within the cylinder are detected and transduced by a piezoelectric accelerometer attached to. the Plexiglas base, digitized and stored by a computer. Beginning at the stimulus onset, 65 readings of 1 ms duration are recorded to obtain the animal's startle amplitude.

Drugs. STEP inhibitors are tested in comparison to vehicle, and clozapine as a positive control.

Prepulse inhibition. Twelve naive mice are tested. Each session is initiated with a 5-min acclimation period followed by five successive 110 dB trials. These trials are not included in the analysis. Six different trial types are then presented: startle pulse (ST110, 110 dB/40 ms), low prepulse stimulus given alone (P74, 74 dB/20 ms), high prepulse stimulus given alone (P90, 90 dB/20 ms), P74 or P90 given 100 ms before the onset of the startle pulse (PP74 and PP90, respectively), and finally a trial where only the background noise is presented (NST) in order to measure the baseline movement in the cylinders. All trials are applied 10 times and presented in random order (P74 and P90 were only given 5 times) and the average inter-trial interval (ITI) was 15 s (10-20 s).

Startle habituation. Twelve naive mice are used in this experiment. Following a 5-min acclimation period, a defined number of trials of 110 dB are presented over a 45-min test session. The intertrial interval varied randomly from 10 to 20 s, with an average of 15 s. The data from the first trial are analyzed separately, because the startle responses to the first stimulus presentation is considered to reflect initial reactivity to a unique event. The remaining trials are grouped in blocks of ten trials each. The amount of habituation (percent habituation) is calculated by the following equation:
100·[(mean amplitude startle for block 1−mean amplitude startle for block11)]/mean amplitude startle for block 1.
A high percentage value reflects a high degree of habituation.

Effects of antipsychotics on PPI in C57BL/6J mice. Separate groups of animals receive an injection of clozapine (0.3, 1, 3 and 30 mg/kg) or STEP modulating agent antagonist (0, 1, 0.3 and 1 mg/kg,) and are tested 30 min later, using the above procedures.

Statistical analysis. Analysis of data is carried out with one-way or two-way ANOVA followed by Duncan test for post-hoc comparisons whenever the ANOVAs indicated statistically significant main or interaction effects. The startle and % PPI are analyzed with a two-way ANOVA with strain (or drug dose) as the between-subject factor and the stimuli as the repeated measure. The analysis of the startle habituation over the session is carried out using two-way ANOVA with strain as the between-subject factor and block as the repeated measure (11 levels). The percent startle habituation is analyzed with one-way ANOVA with the strain as between-subject factor.

### Example 9

### Amphetamine Induced Hyperactivity

d-Amphetamine-induced hyperactivity: C57BL/6J mice, aged 5-6 weeks are used. Hyperactivity is induced by s.c. administration of d-amphetamine sulphate, at a dose of 4 mg/kg, 30 min before testing. Clozapine or STEP modulator plus vehicle is administered i.p/icv. 30 min prior to d-amphetamine. For testing, each mouse is placed into an open-field cage and locomotor activity and stereotyped behavior is recorded for 10 min. The minimal active dose, defined as the lowest dose which significantly inhibits d-amphetamine-induced hyperactivity, is calculated using the Mann-Whitney U-test 2-tailed test.

### Example 10

### Reversal of Phencyclidine (PCP)-Induced Locomotor Hyperactivity

Phencyclidine is known as a psychotomimetic agent, it produces behavioral alterations in animals, which have many characteristics in common with schizophrenia.

Sprague-Dawley derived male rats undergo intracerebroventricular cannula implantation surgery to allow for administration of test compounds directly to the brain parenchyma. Following surgery rats are allowed to recover for a minimum of 5 days. Following recovery period, rats are tested for reversal of PCP induced locomotor hyperactivity. Rats receive a pretreatment of a STEP modulator, administered ICV, ten minutes prior to receiving 5 mg/kg PCP, via interperitoneal injection. Immediately following the PCP treatment, rats are measured for a duration of 60 minutes for their locomotor response in an automated infrared photobeam monitoring apparatus. Test groups include the STEP modulator at a range of active doses, a vehicle control group and a positive control group (clozapine 2 mg/kg, administered subcutaneously). Experiments are performed by routes of administration other than ICV depending on pharmacokinetic properties.

1. A method for identifying a therapeutic agent for treatment of schizophrenia, the method comprising:
detecting the ability of an agent to inhibit the phosphatase activity of a STEP isoform on a substrate or to inhibit the binding of the STEP to NMDA-R, thereby
identifying an inhibitor that is useful as a therapeutic agent,
wherein inhibition of STEP increases NMDA-R signaling activity and is therapeutic in the treatment of schizophrenia.

2. The method according to claim 1, wherein said agent modulates the dephosphorylation by STEP of a protein kinase in the NMDA-R signaling pathway.

3. The method according to claim 2, wherein said kinase is Src.

4. The method according to claim 2, wherein said kinase is Fyn.

5. The method according to claim 2, wherein said kinase is ERK.

6. The method of claim 1, wherein the STEP isoform is human.

7. The method of claim 1, wherein the inhibitor is identified by detecting its ability to inhibit the phosphatase activity of the STEP isoform.

8. The method of claim 1, wherein the inhibitor is identified by detecting its ability to inhibit the binding of the STEP isoform to the NMDA-R.

9. The method according to claim 1, wherein the inhibitor is identified by detecting its ability to modulate the dephosphorylation of NMDA-R by STEP.

10. A method for treating a neurologic disorder disease associated With abnormal NMDA-R-signaling, comprising administering a modulator of a STEP activity, thereby modulating the level of tyrosine phosphorylation of NMDA-R.

11. The method according to claim 10, wherein said neurologic disorder is a psychotic disorder.

12. The method according to claim 11, wherein said psychotic disorder is schizophrenia.

13. The method according to claim 10, wherein said inhibitor modulates the ability of STEP to dephosphorylate a protein kinase in the NMDA-R signaling pathway.

14. The method according to claim 13, wherein said kinase is Src.

15. The method according to claim 13, wherein said kinase is Fyn.

16. The method according to claim 13, wherein said kinase is ERK.

17. The method of claim 10, wherein the inhibitor modulates the ability of STEP to directly or indirectly dephosphorylate NMDA-R.

18. The method of claim 10, wherein the inhibitor modulates the ability of STEP to bind to NMDA-R.

19. The method of claim 10, wherein the neurological disease is selected from the group consisting of ischemic stroke; head trauma or brain injury; Huntington's disease; Parkinson's disease; spinocerebellar degeneration; motor neuron diseases; epilepsy; neuropathic pain; chronic pain; alcohol tolerance; schizophrenia; Alzheimer's disease; dementia; psychosis; drug addiction; ethanol sensitivity, mild cognitive impairment; and depression.

20. A method for identifying a therapeutic agent for treatment of schizophrenia, the method comprising:
contacting a test agent with a cell culture;
detecting the surface expression of NMDA-R subunits in said cell culture contacted with said test agent;
detecting the surface expression of NMDA-R subunits on a control cell culture;
comparing surface expression of said NMDA-R subunits between cell culture contacted with said test agents and said control cell culture, thereby
identifying a test agent that increases surface expression of said NMDA-R subunits;
wherein inhibition of STEP increases NMDA-R surface expression and is therapeutic in the treatment of schizophrenia.
